(19) United States
(12) Patent Application Publication
(10) Pub. No.: US 2025/0281120 A1
(43) Pub. Date: Sep. 11, 2025

(54) WEARABLE ELECTRONIC DEVICE AND METHOD FOR OPERATING SAME

(71) Applicant: Samsung Electronics Co., Ltd., Suwon-si (KR)

(72) Inventors: Sukwang LIM, Suwon-si (KR); Hyoujoo KWON, Suwon-si (KR); Hongji LEE, Suwon-si (KR)

(21) Appl. No.: 19/216,070

(22) Filed: May 22, 2025

Related U.S. Application Data
(63) Continuation of application No. PCT/KR2023/019381, filed on Nov. 28, 2023.

(30) Foreign Application Priority Data
Nov. 29, 2022 (KR) 10-2022-0162980
Feb. 28, 2023 (KR) 10-2023-0026964

(51) Int. Cl. A61B 5/00; A61B 5/01; A61B 5/11

(52) U.S. Cl. CPC A61B 5/6844; A61B 5/01; A61B 5/11

(57) ABSTRACT

A wearable electronic device is provided. The wearable device includes an electrical proximity sensor configured to generate a first biometric signal of a user, an optical proximity sensor configured to generate a second biometric signal of the user, a motion sensor configured to generate a motion sensing signal by sensing a motion of the wearable electronic device, a first temperature sensor configured to generate a third biometric signal by measuring a temperature of the user or an object, a second temperature sensor configured to generate a device temperature signal by measuring an internal temperature of the wearable electronic device, memory storing one or more computer programs, and one or more processors communicatively coupled to the electrical proximity sensor, the optical proximity sensor, the motion sensor, the first temperature sensor, and the second temperature sensor, wherein the one or more computer programs include computer-executable instructions that, when executed by the one or more processors individually or collectively, cause the wearable electronic device to determine whether the wearable electronic device is worn on a body of the user based on the first biometric signal, the second biometric signal, the third biometric signal, and the motion sensing signal, and control a function execution of the wearable electronic device based on whether the wearable electronic device is worn.

### CROSS-REFERENCE TO RELATED APPLICATION(S)

This application is a continuation application, claiming priority under 35 U.S.C. § 365(c), of an International application No. PCT/KR2023/019381, filed on Nov. 28, 2023, which is based on and claims the benefit of a Korean patent application number 10-2022-0162980, filed on Nov. 29, 2022, in the Korean Intellectual Property Office, and of a Korean patent application number 10-2023-0026964, filed on Feb. 28, 2023, in the Korean Intellectual Property Office, the disclosure of each of which is incorporated by reference herein in its entirety.

### BACKGROUND

### 1. Field

The disclosure relates to a wearable electronic device that controls function execution by determining whether a wearable electronic device (e.g., smart watch) is worn and a method of operating the same.

### 2. Description of Related Art

A wearable electronic device (e.g., smart watch) is an electronic device that may be worn on a body of a user due to its miniaturization and lightweight. The wearable electronic device is highly portable, which may lead to improving the convenience of use. The wearable electronic device has high proximity to the body of the user, so may be used for a variety of purposes. The wearable electronic device may include a plurality of sensors (e.g., proximity sensor, temperature sensor, and biometric sensor) to measure biometric information. The wearable electronic device is worn close to the body of the user and thus, may acquire biometric information from the body of the user using the plurality of sensors. The biometric information may include information, such as heart rate, blood pressure, oxygen saturation, blood sugar, skin temperature, and/or body temperature. The acquired biometric information may be displayed for the user or may be transmitted to another electronic device over a network to be utilized for health management of the user. The wearable electronic device may use sensors to identify proximity (or contact) to the skin and may determine whether the wearable electronic device is worn. As a method of identifying whether the wearable electronic device is close to the skin of the user, an optical sensor using the skin's light-reflecting feature and a proximity sensor using the skin's electrical feature may be used. The optical sensor and the proximity sensor may be used alone or together to determine whether the wearable electronic device is worn.

The above information is presented as background information only to assist with an understanding of the disclosure. No determination has been made, and no assertion is made, as to whether any of the above might be applicable as prior art with regard to the disclosure.

### SUMMARY

An optical sensor includes a light emitting portion (e.g., light emitting diode (LED)) configured to emit light and a light receiving portion (e.g., photodiode (PD)) configured to receive light, and may determine a proximity status by irradiating light and by detecting intensity of the reflected light. While most objects absorb light in the infrared wavelength range, skin cells reflect much of it and absorption and reflection of light may be used to determine whether it is worn. A proximity sensor using the skin's electrical feature may include an electrode and an electric circuit, and may determine a contact based on a difference in capacitance or flow of microcurrent that occurs when the skin comes into contact with the electrode. In addition to the proximity sensor, a motion sensor for detecting a motion of a device to increase accuracy of wear detection and a photoplethysmogram (PPG) for detecting a change in a person's blood flow may be additionally used.

In the case of a wear detection method using the proximity sensor, there are cases in which it is not possible to determine whether it is worn depending on a feature of a sensor, so there is a limit in completely recognizing the person's wear status. In the case of an optical proximity sensor (e.g., optical sensor), the feature that the skin reflects infrared ray (IR) is used. Since not all objects absorb IR, an error may occur in determining a wear status for an object that reflects IR. Even in the case of the proximity sensor using the electrical feature, misrecognition may occur when it comes into contact with a material in which electricity flows or an object wet with water. Also, since the electrical feature is generated due to contact, misrecognition may occur when non-contact occurs due to motion, such as a device fixed by a strap, for example, a smart watch. To solve this problem, two methods are interchangeably used, but there is a limit to completely overcoming all exception cases. For example, a bright cloth soaked in water or a light-colored metal in which electricity flows may be mistaken as if the proximity sensor is worn.

To improve misrecognition of a wear status, biometric signals may be acquired using a motion sensor and an optical sensor configured to detect a motion of a wearable electronic device (e.g., smart watch). Whether the wearable electronic device is worn may be relatively accurately determined with a method of using features of wearing situations with a lot of motion and features of blood flow that changes due to heart rate. However, in the case of the motion sensor, data needs to be collected over a long period of time to distinguish between situations, such as sleeping, video viewing, and reading, in which there is extreme lack of human motion. In the case of acquiring biometric signals using the optical sensor, a minute change in biometric signals needs to be detected, so it may be vulnerable to an external environment by vibration or external light while left unattended. Also, the wearable electronic device may be worn with the strap loosely tightened, or a lot of noise may be contained in the acquired signal due to motion. In this case, a considerable amount of time may be used on noise filtering and signal analysis to determine whether the wearable electronic device is worn or not worn.

Aspects of the disclosure are to address at least the above-mentioned problems and/or disadvantages and to provide at least the advantages described below. Accordingly, an aspect of the disclosure is to provide a wearable electronic device that improves misrecognition of detecting whether the wearable electronic device (e.g., smart watch) is worn and may reduce an amount of time required to determine whether it is worn, and a method of operating the same.

Another aspect of the disclosure is to provide a wearable electronic device that improves accuracy of determining whether the wearable electronic device (e.g., smart watch) is worn based on body temperature maintenance and skin temperature change features using a body temperature sensor, and a method of operating the same.

Another aspect of the disclosure is to provide a wearable electronic device that controls function execution by determining whether the electronic device (e.g., smart watch) is worn, and a method of operating the same.

Additional aspects will be set forth in part in the description which follows and, in part, will apparent from the description, or may be learned by practice of the presented embodiments.

In accordance with an aspect of the disclosure, a wearable electronic device is provided. The wearable electronic device includes an electrical proximity sensor configured to generate a first biometric signal of a user, an optical proximity sensor configured to generate a second biometric signal of the user, a motion sensor configured to generate a motion sensing signal by sensing motion of the wearable electronic device, a first temperature sensor configured to generate a third biometric signal by measuring a temperature of the user or an object, a second temperature sensor configured to generate a device temperature signal by measuring an internal temperature of the wearable electronic device, memory storing one or more computer programs, and one or more processors communicatively coupled to the electrical proximity sensor, the optical proximity sensor, the motion sensor, the first temperature sensor, and the second temperature sensor, wherein the one or more computer programs include computer-executable instructions that, when executed by the one or more processors individually or collectively, cause the wearable electronic device to determine whether the wearable electronic device is worn on a body of the user based on the first biometric signal, the second biometric signal, the third biometric signal, and the motion sensing signal, and control a function execution of the wearable electronic device based on whether the wearable electronic device is worn.

In accordance with another aspect of the disclosure, a method performed by a wearable electronic device is provided. The method includes operating, by the wearable electronic device, an electrical proximity sensor to generate a first biometric signal of a user, operating, by the wearable electronic device, an optical proximity sensor to generate a second biometric signal of the user, operating, by the wearable electronic device, a motion sensor to generate a motion sensing signal by sensing motion of the wearable electronic device, operating, by the wearable electronic device, a first temperature sensor to generate a third biometric signal by measuring a temperature of the user or an object, operating, by the wearable electronic device, a second temperature sensor to generate a device temperature signal by measuring an internal temperature of the wearable electronic device, determining, by the wearable electronic device, whether the wearable electronic device is worn on a body of the user based on the first biometric signal, the second biometric signal, the third biometric signal, and the motion sensing signal, and controlling, by the wearable electronic device, a function execution of the wearable electronic device based on whether the wearable electronic device is worn.

In accordance with another aspect of the disclosure, one or more non-transitory computer-readable recording media storing one or more computer programs including computer-executable instructions that, when executed by one or more processors of a wearable electronic device individually or collectively, cause the wearable electronic device to perform operations are provided. The operations include operating, by the wearable electronic device, an electrical proximity sensor to generate a first biometric signal of a user, operating, by the wearable electronic device, an optical proximity sensor to generate a second biometric signal of the user, operating, by the wearable electronic device, a motion sensor to generate a motion sensing signal by sensing motion of the wearable electronic device, operating, by the wearable electronic device, a first temperature sensor to generate a third biometric signal by measuring a temperature of the user or an object, operating, by the wearable electronic device, a second temperature sensor to generate a device temperature signal by measuring an internal temperature of the wearable electronic device, determining, by the wearable electronic device, whether the wearable electronic device is worn on a body of the user based on the first biometric signal, the second biometric signal, the third biometric signal, the device temperature signal, and the motion sensing signal, and controlling, by the wearable electronic device, a function execution of the wearable electronic device based on whether the wearable electronic device is worn.

A wearable electronic device (e.g., smart watch) and a method of operating the same according to an embodiment of the disclosure improves misrecognition of wear detection and reduces an amount of time required to determine whether it is worn.

A wearable electronic device (e.g., smart watch) and a method of operating the same according to an embodiment of the disclosure improves accuracy of determining whether the wearable electronic device (e.g., smart watch) is worn based on body temperature maintenance and skin temperature change features using a body temperature sensor.

A wearable electronic device (e.g., smart watch) and a method of operating the same according to an embodiment of the disclosure controls a function execution by determining whether the electronic device (e.g., smart watch) is worn.

A wearable electronic device (e.g., smart watch) and a method of operating the same according to an embodiment of the disclosure identifies whether the wearable electronic device in which usage time is important is worn and reduces power consumption by deactivating (off) unnecessary functions, such as exercise, health, and notification functions, when a user is not wearing the wearable electronic device.

In a wearable electronic device (e.g., smart watch) and a method of operating the same according to an embodiment of the disclosure, when a user wears the wearable electronic device, the wearable electronic device detects exercise and activates (on) a sensor for collecting biometric signals. Through this, it is possible to activate preset various functions, such as the user's heart rate, sleep, and stress.

A wearable electronic device (e.g., smart watch) and a method of operating the same according to an embodiment of the disclosure activates or deactivates functions of always-on watch and notification delivery while wearing, depending on whether the wearable electronic device is worn.

A wearable electronic device (e.g., smart watch) and a method of operating the same according to an embodiment of the disclosure implements functions for personal information protection using information regarding whether the wearable electronic device is worn. Depending on whether the wearable electronic device is worn, it is possible to implement security functions by locking the wearable electronic device to prevent key functions from operating, and to implement safer security functions using this information.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram illustrating an electronic device in a network environment according to an embodiment of the disclosure;

FIG. 2 is a block diagram illustrating a wearable electronic device (e.g., smart watch) according to an embodiment of the disclosure;

FIG. 3 illustrates a first surface (e.g., rear surface, surface in contact with human skin) of a wearable electronic device (e.g., smart watch) according to an embodiment of the disclosure;

FIG. 4 illustrates a method of measuring a user's skin temperature and body temperature using a temperature sensor according to an embodiment of the disclosure;

FIG. 5 illustrates a temperature sensor provided to a wearable electronic device (e.g., smart watch) according to an embodiment of the disclosure;

FIG. 6 is a flowchart illustrating a method of operating a wearable electronic device (e.g., smart watch) according to an embodiment of the disclosure;

FIG. 7 is a flowchart illustrating a method of operating a wearable electronic device (e.g., smart watch) according to an embodiment of the disclosure;

FIG. 8 is a flowchart illustrating a method of determining temperature equilibrium of a wearable electronic device (e.g., smart watch) according to an embodiment of the disclosure;

FIG. 9 illustrates a change in the temperature when a wearable electronic device (e.g., smart watch) is in contact with the human skin and in contact with an object according to an embodiment of the disclosure;

FIG. 10 is a flowchart illustrating a method of operating a wearable electronic device (e.g., smart watch) according to an embodiment of the disclosure; and

FIG. 11 is a flowchart illustrating a method of operating a wearable electronic device (e.g., smart watch) according to an embodiment of the disclosure.

Throughout the drawings, it should be noted that like reference numerals are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of disclosure is provided for illustration purpose only and not for the purpose of limiting the disclosure as defined in the appended claims and their equivalents.

It is to be understood that the singular forms “a,” “an,” and “the” include plural referents unless the context clearly dictates otherwise. Thus, for example, reference to “a component surface” includes reference to one or more of such surfaces.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi™ chip, a Bluetooth™ chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

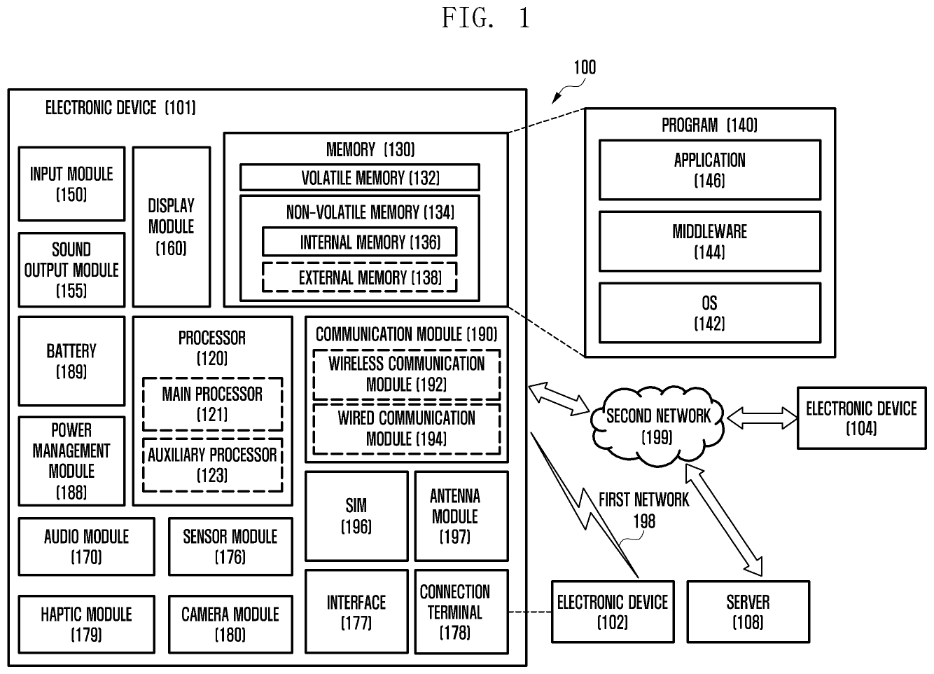

FIG. 1 is a block diagram illustrating an electronic device in a network environment according to an embodiment of the disclosure.

Referring to FIG. 1, an electronic device 101 in a network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connection terminal 178, a haptic module 179, a camera module 180, a power management module 188, a battery 189, a communication module 190, a subscriber identification module (SIM) 196, or an antenna module 197. In some embodiments, at least one of the components (e.g., the connection terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., the electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

The connection terminal 178 may include a connector via which the electronic device 101 may be physically connected with the external electronic device (e.g., the electronic device 102). According to an embodiment, the connection terminal 178 may include, for example, an HDMI connector, a USB connector, an SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi™) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 gigabits per second (Gbps) or more) for implementing eMBB, loss coverage (e.g., 164 decibels (dB) or less) for implementing mMTC, or U-plane latency (e.g., 0.5 milliseconds (ms) or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices (e.g., electronic devices 102 and 104 and the server 108). For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

According to an embodiment, the display module 160 shown in FIG. 1 may include a bar type or plate type display (e.g., organic light emitting diode (OLED) display).

According to an embodiment, the display module 160 shown in FIG. 1 may include a flexible display (e.g., flexible OLED display) configured to fold or unfold a screen (e.g., display screen).

According to an embodiment, the display module 160 shown in FIG. 1 may include a flexible display (e.g., flexible OLED display) that is slidably arranged to provide a screen (e.g., display screen).

According to an embodiment, the electronic device 101 shown in FIG. 1 may include a wearable electronic device, a smart watch, an augmented reality (AR) electronic device, a virtual reality (VR) electronic device, a mobile phone (e.g., smart phone), a laptop personal computer (PC), a tablet PC, and/or an audio electronic device (e.g., wired earphones, wireless earphones).

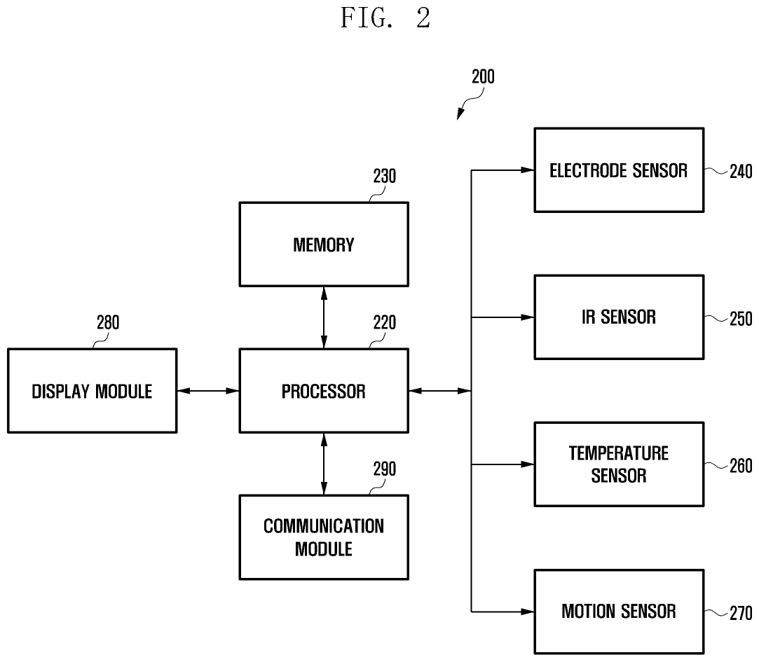

According to an embodiment, the sensor module 176 may include an electrical proximity sensor (e.g., electrode sensor 240 of FIG. 2).

According to an embodiment, the sensor module 176 may include an optical proximity sensor (e.g., optical sensor) (e.g., photoplethysmogram (PPG) sensor) (e.g., infrared ray (IR) sensor 250 of FIG. 2).

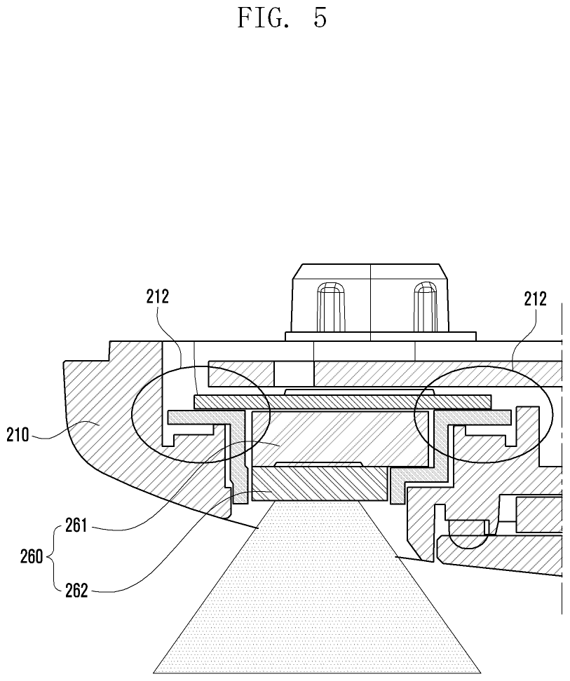

According to an embodiment, the sensor module 176 may include a temperature sensor (e.g., temperature sensor 260 of FIGS. 2 and 5) that may measure the user's skin temperature and body temperature.

According to an embodiment, the sensor module 176 may include a motion sensor (e.g., motion sensor 270 of FIG. 2) that may detect motion of the electronic device 101.

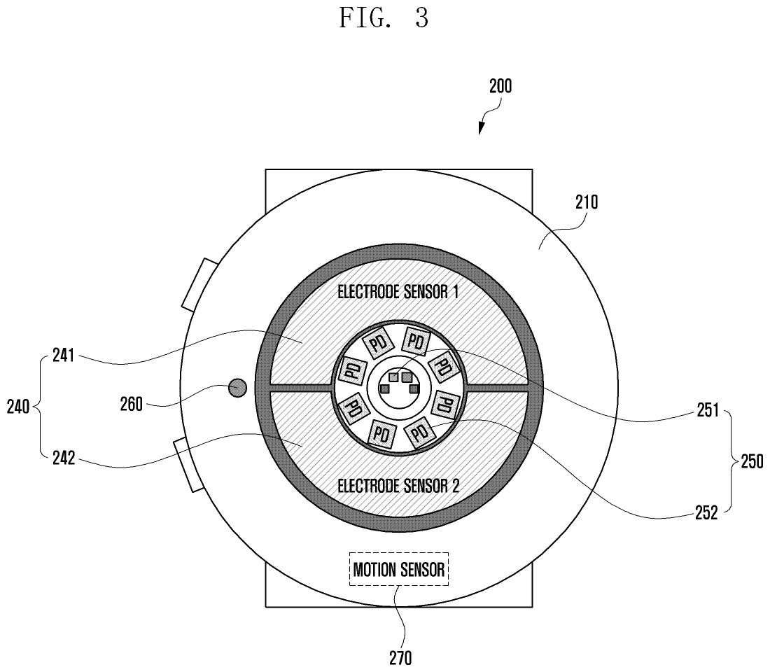

According to an embodiment, the electrical proximity sensor (e.g., electrode sensor 240 of FIG. 2), the optical proximity sensor (e.g., optical sensor) (e.g., IR sensor 250 of FIG. 2), the temperature sensor (e.g., temperature sensor 260 of FIGS. 2 and 5), and the motion sensor (e.g., motion sensor 270 of FIG. 2) may be provided to the electronic device 101 (e.g., wearable electronic device 200 of FIGS. 2 and 3).

FIG. 2 is a block diagram illustrating a wearable electronic device (e.g., smart watch) according to an embodiment of the disclosure.

FIG. 3 illustrates a first surface (e.g., rear surface, surface in contact with human skin) of a wearable electronic device (e.g., smart watch) according to an embodiment of the disclosure.

Referring to FIGS. 2 and 3, a wearable electronic device 200 (e.g., smart watch) (e.g., electronic device 101 of FIG. 1) according to an embodiment of the disclosure may include a processor 220 (e.g., processor 120 of FIG. 1), memory 230 (e.g., memory 130 of FIG. 1), an electrode sensor 240 (e.g., electrical proximity sensor), an infrared ray (IR) sensor 250 (e.g., optical proximity sensor), a temperature sensor 260 (e.g., temperature sensor 260 of FIG. 5), a motion sensor 270, a display module 280 (e.g., display module 160 of FIG. 1), and a communication module 290 (e.g., communication module 190 of FIG. 1).

According to an embodiment, the wearable electronic device 200 according to an embodiment of the disclosure may include a program (e.g., program 140 of FIG. 1) for operating an electronic device and a battery (e.g., battery 189 of FIG. 1) configured to supply power for operating each of components. For example, the battery 189 may include a non-rechargeable primary cell, a rechargeable secondary cell, or a fuel cell. The battery 189 may be integrally provided inside the wearable electronic device 200, or may be detachably provided to the wearable electronic device 200.

The wearable electronic device 200 according to an embodiment of the disclosure may include a first surface (e.g., rear surface, surface in contact with human skin), a second surface (e.g., surface on which the screen of the display module 280 is displayed), and a third surface (e.g., side surface) provided to encompass space between the first surface (e.g., rear surface, surface in contact with human skin) and the second surface (e.g., surface on which the screen of the display module 280 is displayed).

The processor 220, the memory 230, the electrode sensor 240 (e.g., electrical proximity sensor), the IR sensor 250 (e.g., optical proximity sensor), the temperature sensor 260, the motion sensor 270, the display module 280, and the communication module 290 may be provided in space formed by a housing 210 of the wearable electronic device 200.

According to an embodiment, the electrode sensor 240 (e.g., electrical proximity sensor), the IR sensor 250 (e.g., optical proximity sensor), and the temperature sensor 260 (e.g., contact-free temperature sensor) may be provided to face the first surface (e.g., rear surface, surface in contact with human skin) of the wearable electronic device 200 according to an embodiment of the disclosure.

For example, the electrode sensor 240 (e.g., electrical proximity sensor), the IR sensor 250 (e.g., optical proximity sensor), and the temperature sensor 260 (e.g., contact-free temperature sensor) may be provided to be adjacent to a rear plate of the wearable electronic device 200.

According to an embodiment, the display module 280 may be provided to face the second surface (e.g., surface on which the screen is displayed) of the wearable electronic device 200 according to an embodiment of the disclosure. For example, the display module 280 may be exposed to be visually visible. A shape of the display module 280 may be formed to correspond to a frontal shape of the housing 210. The display module 280 may be formed in a circular, oval, or polygonal shape. For example, the display module 280 may include a touch sensor. Presence or absence of touch and intensity (pressure) of touch may be measured through the touch sensor. The touch sensor may be combined with or provided to be adjacent to a pressure sensor, and/or a fingerprint sensor.

According to an embodiment, the motion sensor 270 may be provided inside the wearable electronic device 200 and may not be viewed from outside.

According to an embodiment, the wearable electronic device 200 according to an embodiment of the disclosure may include a coupling member (not shown) configured to connect to at least a portion of the housing 210, and to detachably couple the wearable electronic device 200 to a portion of the user's body (e.g., wrist). The coupling member may be, for example, a strap that wraps around the wrist of the user to fasten the wearable electronic device 200.

According to an embodiment, the processor 220 of the wearable electronic device 200 (e.g., smart watch) according to an embodiment of the disclosure may include one or more of a central processing unit, an application processor, a graphic processing unit (GPU), an application processor, a sensor processor, and a communication processor. For example, the processor 220 may control the operation of the communication module 290 to operate in conjunction with an external electronic device and/or an external wearable electronic device.

According to an embodiment, the memory 230 of the wearable electronic device 200 (e.g., smart watch) according to an embodiment of the disclosure may include volatile memory and/or nonvolatile memory. For example, the memory 230 may include instructions for operation performance of the processor 220. Also, the memory 230 may include instructions for operation performance of the electrode sensor 240, the IR sensor 250, the temperature sensor 260, the motion sensor 270, the display module 280, and/or the communication module 290.

According to an embodiment, the wearable electronic device 200 (e.g., smart watch) according to an embodiment of the disclosure may be worn on the user's body (e.g., wrist). The wearable electronic device 200 (e.g., smart watch) may operate in conjunction with the external electronic device (e.g., smart phone, tablet PC, laptop PC) and/or the external wearable electronic device (e.g., audio electronic device, wireless earphones).

According to an embodiment, the processor 220 of the wearable electronic device 200 (e.g., smart watch) according to an embodiment of the disclosure may control the operation of at least one of the electrode sensor 240, the IR sensor 250, the temperature sensor 260, and the motion sensor 270 to acquire the user's motion signals, exercise signals, health-related signals, and biometric signals (e.g., hearth rate, blood pressure, oxygen saturation, blood sugar, skin temperature, and/or body temperature).

According to an embodiment, the processor 220 of the wearable electronic device 200 (e.g., smart watch) according to an embodiment of the disclosure may control function execution of the wearable electronic device 200 (e.g., smart watch) based on at least one of motion signals, exercise signals, health-related signals, and biometric signals (e.g., hearth rate, blood pressure, oxygen saturation, blood sugar, skin temperature, and/or body temperature) acquired by operating the electrode sensor 240, the IR sensor 250, the temperature sensor 260, and the motion sensor 270. For example, the processor 220 may determine whether the wearable electronic device 200 (e.g., smart watch) is worn based on at least one of motion signals, exercise signals, health-related signals, and biometric signals (e.g., hearth rate, blood pressure, oxygen saturation, blood sugar, skin temperature, and/or body temperature). The processor 220 may selectively turn on or off execution of functions of the wearable electronic device 200 (e.g., smart watch) whether the wearable electronic device 200 (e.g., smart watch) is worn.

According to an embodiment, the electrode sensor 240 (e.g., electrocardiograph (ECG) sensor, electrical wearing sensor, electrical proximity sensor) may include a first electrode 241, a second electrode 242, and a circuit (e.g., integrated circuit (IC)) configured to control operations of the first electrode 241 and the second electrode 242 and to detect biometric signals. For example, the first electrode 241 and the second electrode 242 may form at least a portion of the first surface (e.g., rear surface, surface in contact with human skin) of the wearable electronic device 200 (e.g., smart watch). For example, the first electrode 241 and the second electrode 242 may be electrically connected when both of them are in contact with the skin of the user, and an electrical signal may be acquired from a portion of the user's body through electrical connection of the first electrode 241 and the second electrode 242. The circuit that detects a biometric signal may acquire a biometric signal of the user based on the electrical signal. The biometric signal acquired by the electrode sensor 240 (e.g., ECG sensor, electrical wearing sensor, electrical proximity sensor) may be provided to the processor 220. For example, the circuit that detects a biometric signal may acquire a first biometric signal of the user based on a difference in capacitance that occurs when the first electrode 241 and the second electrode 242 come into contact with the skin of the user. The first biometric signal acquired from the electrode sensor 240) (e.g., ECG sensor, electrical wearing sensor, electrical proximity sensor) may be provided to the processor 220. For example, the processor 220 may determine whether the wearable electronic device 200 (e.g., smart watch) is worn based on the first biometric signal from the electrode sensor 240 (e.g., ECG sensor, electrical wearing sensor, electrical proximity sensor).

According to an embodiment, the IR sensor 250 (e.g., optical proximity sensor) (e.g., optical sensor, PPG sensor) may include a light emitting portion 251 configured with a plurality of LEDs that emit light and a light receiving portion 252 configured with a plurality of photodiodes (PDs) that receive light and convert the same to an electrical signal. For example, the optical sensor (e.g., PPG sensor) may include a plurality of LEDs that irradiate red and green light and a plurality of PDs. The optical sensor (e.g., PPG sensor) may further include the IR sensor 250 that irradiates IR light. Without being limited thereto, the optical sensor (e.g., PPG sensor) may also include one or a plurality of IR LEDs that irradiate IR light without the plurality of LEDs that irradiate red and green light. Using the IR sensor 250 or the optical sensor (e.g., PPG sensor) that includes the IR sensor 250, whether the user is wearing the wearable electronic device 200 (e.g., smart watch) (e.g., whether the wearable electronic device 200 (e.g., smart watch) is close to the skin of the user) may be determined.

For example, the light emitting portion 251 may irradiate light of a wavelength (mainly IR) that the skin reflects to the skin. For example, the plurality of LEDs of the IR sensor 250 (e.g., optical proximity sensor, optical wear sensor) may include a first LED that generates light with a wavelength of about 540 nanometers (nm) (e.g., green light), a second LED that generates light with a wavelength of about 660 nm (e.g., red light), a third LED that that generates light with a wavelength of about 880 nm (e.g., infrared light), and a fourth LED that that generates light with a wavelength of about 940 nm. For example, the light receiving portion 252 may receive light that is emitted from the light emitting portion 251 and reflected from an object or the skin and converts the same to an electrical signal, and may generate a second biometric signal of the user. The second biometric signal acquired from the IR sensor 250 (e.g., optical proximity sensor, optical wear sensor) may be provided to the processor 220. For example, the IR sensor 250 (e.g., optical proximity sensor, optical wear sensor) may acquire the second biometric signal by emitting light to the skin surface on which the blood vessels of the human body are present and by receiving the reflected light of the emitted light. For example, the second biometric signal acquired from the IR sensor 250 (e.g., optical proximity sensor, optical wear sensor) may be provided to the processor 220. For example, the processor 220 may identify biometric information such as the user's heart rate information and/or heart rate variation information based on the second biometric signal, and may determine the user's current state (e.g., sleep, exercise, or normal activity). For example, the processor 220 may determine whether the wearable electronic device 200 (e.g., smart watch) is worn based on the second biometric signal from the IR sensor 250.

According to an embodiment, the temperature sensor 260 (e.g., contact-free temperature sensor) may measure the user's skin temperature or body temperature by reflecting features of electromagnetic waves irradiated from an object according to its temperature. For example, the temperature sensor 260 (e.g., contact-free temperature sensor) may include a contact-free IR temperature sensor. For example, since the temperature inside the sensor may have influence due to features of the contact-free temperature sensor, the temperature sensor 260 (e.g., contact-free temperature sensor) may include a temperature sensor configured to measure the internal temperature (T_{amb}) of the temperature sensor 260. The temperature sensor that measures the internal temperature (T_{amb}) of the wearable electronic device 200 may include a thermistor. The internal temperature of the wearable electronic device 200 may be measured using the thermistor. The temperature sensor may measure the internal temperature (T_{amb}) of the wearable electronic device 200 and may generate a device temperature signal. The device temperature signal generated by the temperature sensor may be provided to the processor 220.

For example, an object may have the temperature of the absolute temperature (−273.15° C.) or more and may reflect electromagnetic waves with a wavelength corresponding to the temperature. As the temperature increases, the wavelength of electromagnetic waves becomes shorter and an amount of radiant energy may increase. The temperature sensor 260 (e.g., contact-free temperature sensor) may detect the temperature using the Seebeck effect that electromotive force is generated according to a difference in the temperature between a hot junction and a cold junction of an internal thermopile.

For example, the temperature sensor 260 (e.g., contact-free temperature sensor) may acquire a third biometric signal according to the user's skin temperature or body temperature. The third biometric signal may include data of the user's skin temperature or body temperature. For example, the third biometric signal acquired from the temperature sensor 260 (e.g., contact-free temperature sensor) may be provided to the processor 220. For example, the processor 220 may determine whether the wearable electronic device 200 (e.g., smart watch) is worn based on the third biometric signal from the temperature sensor 260 (e.g., contact-free temperature sensor).

According to an embodiment, the motion sensor 270 may detect a state (or posture, direction), motion, and inertia of the wearable electronic device 200 (e.g., smart watch). For example, the motion sensor 270 may detect velocity, acceleration, angular velocity, and/or angular acceleration by the motion of the wearable electronic device 200 (e.g., smart watch). The motion sensor 270 may generate a motion detection signal for the motion of the wearable electronic device 200 (e.g., smart watch) and may provide the motion detection signal to the processor 220. For example, the processor 220 may determine the user's current state (e.g., sleep, exercise, or normal activity) based on the motion detection signal from the motion sensor 270. The processor 220 may control execution of functions of the wearable electronic device 200 (e.g., smart watch) according to the user's current state (e.g., sleep, exercise, or normal activity). For example, the wearable electronic device 200 (e.g., smart watch) may include a 6-axis sensor (e.g., acceleration sensor and gyro sensor). The 6-axis sensor may sense whether the user is in motion and the magnitude of the motion. A motion sensing value of the 6-axis sensor may be provided to the processor 220. The processor 220 may determine whether there was the motion of the user and the magnitude of the motion based on the motion sensing value from the 6-axis sensor. For example, the wearable electronic device 200 (e.g., smart watch) may include a global positioning system (GPS) sensor to detect a current location. When the wearable electronic device 200 (e.g., smart watch) includes the GPS sensor, the processor 220 may control function execution of the wearable electronic device 200 (e.g., smart watch) based on acquired location information.

According to an embodiment, the wearable electronic device 200 (e.g., smart watch) according to an embodiment of the disclosure may generate sensing signals to determine whether it is worn using the electrode sensor 240 (e.g., ECG sensor, electrical proximity sensor, electrical wearing sensor), the IR sensor 250 (e.g., optical proximity sensor, optical wear sensor), and the temperature sensor 260. The processor 220 may determine whether the wearable electronic device 200 (e.g., smart watch) is worn based on sensing signals acquired from the electrode sensor 240 (e.g., ECG sensor, electrical proximity sensor, electrical wearing sensor), the IR sensor 250 (e.g., optical proximity sensor, electrical wearing sensor), and/or the temperature sensor 260.

For example, the processor 220 may determine whether the wearable electronic device 200 (e.g., smart watch) is worn using all three sensors, the electrode sensor 240 (e.g., ECG sensor, electrical proximity sensor, electrical wearing sensor), the IR sensor 250 (e.g., optical proximity sensor, electrical wearing sensor), and the temperature sensor 260 (e.g., first temperature sensor that measures person's skin temperature, second temperature sensor (thermistor) that measures temperature inside device).

For example, the processor 220 may determine whether the user is wearing the wearable electronic device 200 (e.g., smart watch) using both sensors, the electrode sensor 240 (e.g., ECG sensor, electrical proximity sensor, electrical wearing sensor) and the IR sensor 250) (e.g., optical proximity sensor, optical wear sensor).

For example, the processor 220 may determine whether the wearable electronic device 200 (e.g., smart watch) is worn using one of the electrode sensor 240 (e.g., ECG sensor, electrical proximity sensor, electrical wearing sensor), the IR sensor 250 (e.g., optical proximity sensor, optical wear sensor), and the temperature sensor 260.

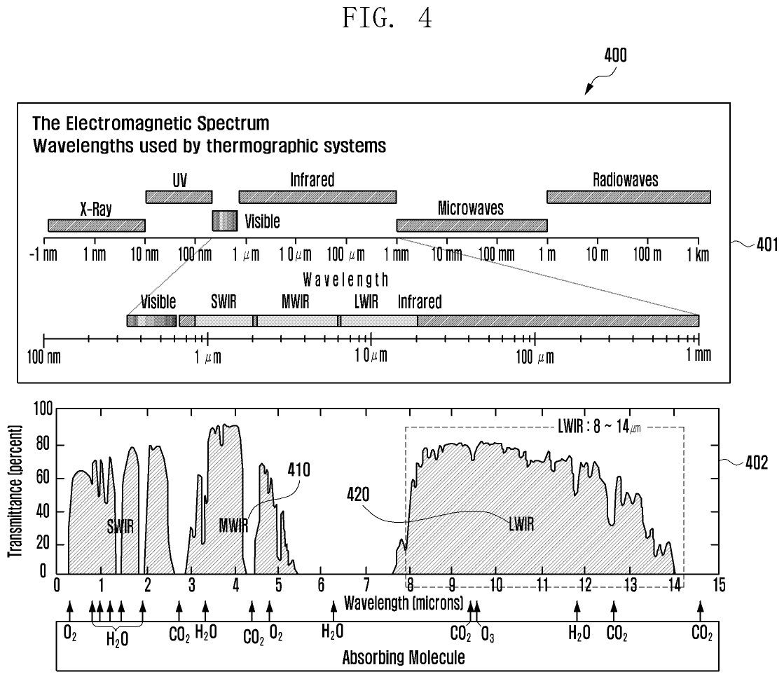

FIG. 4 is a view illustrating a method of measuring a user's skin temperature and body temperature using a temperature sensor according to an embodiment of the disclosure. FIG. 5 illustrates a temperature sensor provided to a wearable electronic device (e.g., smart watch) according to an embodiment of the disclosure.

Referring to FIGS. 4 and 5, a temperature sensor 260 (e.g., contact-free temperature sensor) may include a temperature sensor portion 261 and a lens portion 262 (e.g., silicon lens) configured such that field of view (FOV) of the temperature sensor 260 (e.g., infrared temperature sensor, contact-free temperature sensor) may be satisfied. For example, a wearable electronic device (e.g., wearable electronic device 200 of FIGS. 2 and 3) according to an embodiment of the disclosure may include the temperature sensor 260 (e.g., infrared temperature sensor, contact-free temperature sensor) provided in the housing 210, and a sealing structure 212 (e.g., waterproof structure) at a location at which the temperature sensor 260 (e.g., infrared temperature sensor, contact-free temperature sensor) is provided. Dustproofing and waterproof may be achieved at a location at which the temperature sensor 260 (e.g., infrared temperature sensor, contact-free temperature sensor) is provided through the sealing structure 212 (e.g., waterproof structure). Also, the FOV of the light receiving portion of the temperature sensor 260 (e.g., infrared temperature sensor, contact-free temperature sensor) may be secured to be 80% or more of the FOV of the temperature sensor 260 (e.g., infrared temperature sensor, contact-free temperature sensor) itself.

Referring to FIG. 4, according to an embodiment, view 400 illustrates that graph 401, shown at the top in FIG. 4, shows the electromagnetic spectrum that is decomposed and arranged according to wavelengths. The electromagnetic spectrum may be arranged into a total of 7 sections: gamma rays, X-rays, ultraviolet radiation, visible light, infrared radiation, radio waves, and microwaves, and roughly shows to which section an infrared portion corresponds according to wavelengths. Graph 402 of the wavelength spectrum shown at the bottom of FIG. 4 generally divides infrared radiation into three, short-wavelength infrared (SWIR), mid-wavelength infrared (MWIR), and long wave infrared (LWIR) according to wavelengths.

For example, IR energy may be irradiated from an object itself in wavelength areas of short-wavelength infrared (SWIR) (about 1.4 to about 3 micrometers (um)), mid-wavelength infrared (MWIR) (about 3 to about 8 μm) 410, and long wave infrared (about 8 to about 14 μm) 420 bands. The SWIR, the MWIR, and the LWIR shown in FIG. 4 are divided from the infrared according to wavelengths. In infrared radiation, infrared energy and absorbing molecules in the respective SWIR, MWIR, LWIR wavelength bands may be matched. The temperature sensor 260 (e.g., infrared temperature sensor, contact-free temperature sensor) does not require an external light source to detect thermal infrared of an object and may only receive light. The temperature sensor 260 (e.g., infrared temperature sensor, contact-free temperature sensor) may generate a third biometric signal by receiving at least one of wavelengths in the SWIR (about 1.4 to about 3 μm), MWIR (about 3 to about 8 μm) 410, and LWIR (about 8 to 14 μm) 420 bands.

According to an embodiment, the wearable electronic device 200 (e.g., smart watch) may determine whether it is worn using the first biometric signal from the electrode sensor (e.g., electrode sensor 240 of FIG. 2) and the second biometric signal from the IR sensor (e.g., IR sensor 250 of FIG. 2). Here, to reduce errors in determining whether the wearable electronic device 200 (e.g., smart watch) is worn, whether it is worn may be determined by additionally using the third biometric signal from the temperature sensor 260 (e.g., contact-free temperature sensor).

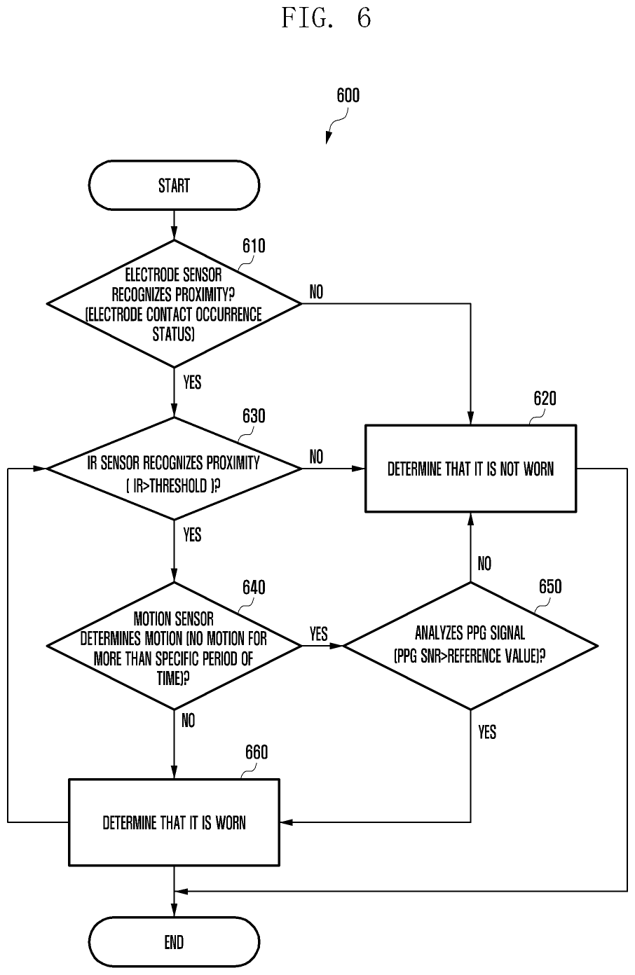

FIG. 6 is a flowchart illustrating a method of operating a wearable electronic device (e.g., smart watch) according to an embodiment of the disclosure.

Referring to FIGS. 2 and 6, flowchart 600 illustrates that, in operation 610, a processor 220 may operate an electrode sensor 240. For example, through operating of the electrode sensor 240, whether the electrode sensor 240 and the skin of the user are close (or in contact) may be identified. For example, through operating of the electrode sensor 240, whether the user is wearing the wearable electronic device 200 may be identified. Through operating of the electrode sensor 240, a first biometric signal may be acquired. For example, the processor 220 may determine whether the wearable electronic device 200 and the skin of the user are close (or in contact) based on the first biometric signal. For example, the processor 220 may determine whether the user is wearing the wearable electronic device 200 based on the first biometric signal.

In case of being determined that the wearable electronic device 200 is not close to (or in contact with) the skin of the user (NO) as a determination result of operation 610, operation 620 may be performed.

In operation 620, the processor 220 may determine that the wearable electronic device 200 is not worn on the user's body (e.g., wrist).

When it is determined that the wearable electronic device 200 is close to (or in contact with) the skin of the user as a determination result of operation 610 (YES), operation 630 may be performed.

In operation 630, the processor 220 may operate the IR sensor 250. Through operating of the IR sensor 250, whether the IR sensor 250 and the skin of the user are close (or in contact) may be identified. Through operating of the IR sensor 250, a second biometric signal may be acquired. The processor 220 may determine whether the wearable electronic device 200 is close to (or contact with) the skin of the user (e.g., user is wearing the wearable electronic device 200) based on the second biometric signal.

For example, the processor 220 may determine whether the second biometric signal (IR) exceeds a threshold and may determine whether the wearable electronic device 200 is close to (or in contact with) the skin of the user (e.g., the user is wearing the wearable electronic device 200). If second biometric signal (IR) does not exceed the threshold (NO), the processor 220 may perform operation 620.

In operation 620, the processor 220 may determine that the wearable electronic device 200 is not worn on the user's body (e.g., wrist).

Meanwhile, if the second biometric signal (IR) exceeds the threshold as a determination result of operation 630, the processor 220 may perform operation 640.

In operation 640, the processor 220 may operate the motion sensor 270. The motion sensor 270 may sense motion of the wearable electronic device 200, and may acquire a motion sensing signal according to the motion. The processor 220 may determine whether there is no motion for more than a specific period of time (e.g., more than preset period of time) based on the motion sensing signal. For example, when the motion of the wearable electronic device 200 is not detected for more than the specific period of time (e.g., more than preset period of time), the wearable electronic device 200 may not be worn of the user's body (e.g., wrist), but may be placed elsewhere (e.g., placed on table) or stored (e.g., stored in drawer or box). In this case, the processor 220 may determine whether the user is wearing the wearable electronic device 200 based on the motion sensing signal.

If it is determined that the wearable electronic device 200 has not moved for more than the specific period of time (e.g., more than preset period of time) (YES) as a determination result of operation 640, operation 650 may be performed.

In operation 650, the processor 220 may operate the IR sensor 250. Through operating of the IR sensor 250, a second biometric signal that includes a photoplethysmogram (PPG) signal may be acquired. The processor 220 may analyze the PPG signal included in the second biometric signal from the IR sensor 250, and may determine whether a PPG signal-to-noise ratio (SNR) exceeds a reference value.

For example, the processor 220 may identify whether a human-like periodic signal (about 50 to about 200 beats per minute (BPM)) is included in the second biometric signal. When the human-like periodic signal (about 50 to about 200 BPM) is included in the second biometric signal, it may be determined that the wearable electronic device 200 is worn on the user's body (e.g., wrist).

For example, when there is no motion of the wearable electronic device 200 for more than the specific period of time (e.g., more than preset period of time), the wearable electronic device 200 may not be worn on the user's body (e.g., wrist), but may be placed elsewhere (e.g., placed on table) or stored (e.g., stored in drawer or box). However, although the user is actually wearing the wearable electronic device 200, there may be no motion for the specific period of time (e.g., preset period of time) (e.g., sleeping state or no or very small body motion state). In this case, whether the wearable electronic device 200 is being worn on the body (e.g., wrist) may be additionally identified using a PPG SNR value.

If the PPG SNR value does not exceed the reference value as a determination result of operation 650, operation 620 may be performed.

In operation 620, the processor 220 may determine that the wearable electronic device 200 is not worn on the user's body (e.g., wrist). For example, when the PPG SNR value does not exceed the reference value (NO), the wearable electronic device 200 may not be worn on the user's body (e.g., wrist), but may be placed elsewhere or may be stored.

Meanwhile, if the motion of the wearable electronic device 200 is detected within the specific period of time (e.g., within preset period of time) (NO) as a determination result of operation 640, operation 660 may be performed.

In operation 660, the processor 220 may determine that the wearable electronic device 200 is worn on the user's body (e.g., wrist).

Meanwhile, if the PPG SNR value exceeds the reference value as a determination result of operation 650, operation 660 may be performed.

In operation 660, the processor 220 may determine that the wearable electronic device 200 is worn on the user's body (e.g., wrist).

At least some of the operations shown in FIG. 6 may be omitted. At least some operations mentioned with reference to other drawings herein may be added and/or inserted before or after at least some operations shown in FIG. 6.

According to an embodiment, operations of the electrode sensor 240, the IR sensor 250, and the motion sensor 270 may be performed in parallel, and determination operations of the processor 220 may be sequentially performed.

According to an embodiment, operations of the electrode sensor 240, the IR sensor 250, and the motion sensor 270 may be sequentially performed, and determination operations of the processor 220 may be sequentially performed.

At least some of the operations shown in FIG. 6 may be omitted. At least some operations mentioned with reference to other drawings herein may be added and/or inserted before or after at least some operations shown in FIG. 6.

According to an embodiment, operations of the electrode sensor 240, the IR sensor 250, and the motion sensor 270 may be performed in parallel, and determination operations of the processor 220 may be sequentially performed.

According to an embodiment, operations of the electrode sensor 240, the IR sensor 250, and the motion sensor 270 may be sequentially performed, and determination operations of the processor 220 may be sequentially performed.

According to an embodiment, the operations shown in FIG. 6 may be performed by the processor 220 (e.g., processor 120 of FIG. 1) of the wearable electronic device 200 (e.g., smart watch). For example, the memory 230 (e.g., memory 130 of FIG. 1) of the wearable electronic device 200 (e.g., smart watch) may include instructions that cause the processor 220 to perform at least some operations shown in FIG. 6 when the processor 220 is executed.

According to an embodiment, the operations shown in FIG. 6 may be performed by a processor (e.g., processor 120 of FIG. 1) of an external electronic device (e.g., smart phone, tablet PC, wearable electronic device). For example, memory (e.g., memory 130 of FIG. 1) of the external electronic device (e.g., smart phone, tablet PC, wearable electronic device) may include instructions that cause the processor 120 to perform at least some operations shown in FIG. 6 when the processor 120 is executed.

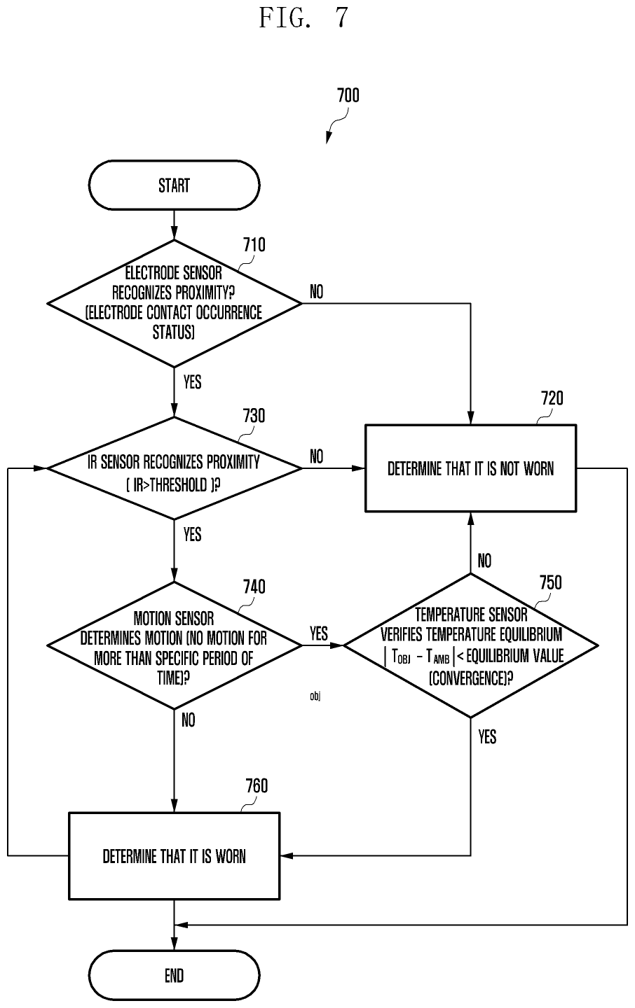

FIG. 7 is a flowchart illustrating a method of operating a wearable electronic device (e.g., smart watch) according to an embodiment of the disclosure.

Referring to FIGS. 2 and 7, flowchart 700 illustrates that, in operation 710, a processor 220 may operate an electrode sensor 240. Through operating of the electrode sensor 240, whether the electrode sensor 240 and the user skin are close (or in contact) may be identified. Through operating of the electrode sensor 240, a first biometric signal may be acquired. The processor 220 may determine whether the wearable electronic device 200 is close to (or in contact with) the skin of the user based on the first biometric signal.

When it is not determined that the wearable electronic device 200 is close to (or in contact with) the skin of the user (NO) as a determination result of operation 710, operation 720 may be performed.

In operation 720, the processor 220 may determine that the wearable electronic device 200 is not worn on the user's body (e.g., wrist).

When it is determined that the wearable electronic device 200 is close to (or in contact with) the skin of the user (YES) as a determination result of operation 710, operation 730 may be performed.

In operation 730, the processor 220 may operate the IR sensor 250 (e.g., optical sensor, optical wear sensor). Through operating of the IR sensor 250, whether the IR sensor 250 and the user skin are close (or in contact) may be identified. Through operating of the IR sensor 250, a second biometric signal may be acquired. The processor 220 may determine whether the wearable electronic device 200 is close to (or in contact with) the skin of the user based on the second biometric signal.

For example, the processor 220 may determine whether the second biometric signal (IR) exceeds a threshold, and may determine whether the wearable electronic device 200 is close to (or in contact with) the skin of the user. If the second biometric signal (IR) does not exceed the threshold (NO), the processor 220 may perform operation 720.

In operation 720, the processor 220 may determine that the wearable electronic device 200 is not worn on the user's body (e.g., wrist).

Meanwhile, if the second biometric signal (IR) exceeds the threshold (YES) as a determination result of operation 730, the processor 220 may perform operation 740.

In operation 740, the processor 220 may operate the motion sensor 270. The motion sensor 270 may sense motion of the wearable electronic device 200, and may acquire a motion sensing signal according to the motion. The processor 220 may determine whether there is no motion for more than a specific period of time (e.g., more than preset period of time) based on the motion sensing signal. For example, when the motion of the wearable electronic device 200 is not detected for more than the specific period of time (e.g., more than preset period of time), the wearable electronic device 200 may not be worn of the user's body (e.g., wrist), but may be placed elsewhere (e.g., placed on table) or stored (e.g., stored in drawer or box). In this case, the processor 220 may determine whether the wearable electronic device 200 is worn on the user's body (e.g., wrist) based on the motion sensing signal.

Meanwhile, if the motion of the wearable electronic device 200 is detected within the specific period of time (e.g., within preset period of time) (NO) as a determination result of operation 740, operation 760 may be performed.

In operation 760, the processor 220 may determine that the wearable electronic device 200 is worn on the user's body (e.g., wrist).

If it is determined whether there is no motion of the wearable electronic device 200 for more than the specific period of time (e.g., more than preset period of time) (YES) as a determination result of operation 740, operation 750 may be performed.

In operation 750, the processor 220 may operate the temperature sensor 260. Through operating of the temperature sensor 260, a third biometric signal may be acquired.

For example, if the temperature is measured when the user is wearing the wearable electronic device 200, the third biometric signal includes data of the user's skin temperature or body temperature. For example, if the temperature is measured when the user is not wearing the wearable electronic device 200, the third biometric signal includes data of the temperature of an object or the ambient temperature of the wearable electronic device 200. The processor 220 may identify whether equilibrium of the measured temperature (user's skin temperature, body temperature or temperature of object) included in the third biometric signal. For example, if the equilibrium of the measured temperature (user's skin temperature, body temperature or temperature of object) is not identified based on temperature equilibrium (Tₓ(=T_{obj}−T_{amb})) data when the wearable electronic device 200 comes into contact with the human skin and comes into contact with the object (NO), the processor 220 may perform operation 720. For example, ‘T_{obj}’ may represent the temperature of a target object, person's skin, based on a sensor. ‘T_{amb}’ may represent the temperature inside the sensor (e.g., temperature of electronic device itself).

In operation 720, the processor 220 may determine that the wearable electronic device 200 is not worn on the user's body (e.g., wrist).

In operation 750, the processor 220 may calculate a difference between the measured temperature (user's skin temperature, body temperature or temperature of object) and the device temperature (e.g., temperature of wearable electronic device 200). The processor 220 may determine whether the measured temperature (user's skin temperature, body temperature or temperature of object) is in an equilibrium state (or converges to equilibrium state) based on the difference between the measured temperature (user's skin temperature, body temperature or temperature of object) and the device temperature (e.g., temperature of wearable electronic device 200). If the equilibrium of the measured temperature (user's skin temperature, body temperature or temperature of object) is identified based on the temperature equilibrium (Tₓ(=T_{obj}−T_{amb})) data when the wearable electronic device 200 comes into contact with the human skin and comes into contact with the object (YES), the processor 220 may perform operation 760.

In operation 760, the processor 220 may determine that the wearable electronic device 200 is worn on the user's body (e.g., wrist). At least some of the operations shown in FIG. 7 may be omitted. At least some operations mentioned with reference to other drawings herein may be added and/or inserted before or after at least some operations shown in FIG. 7.

According to an embodiment, operations of the electrode sensor 240, the IR sensor 250, and the motion sensor 270 may be performed in parallel, and determination operations of the processor 220 may be sequentially performed.

According to an embodiment, operations of the electrode sensor 240, the IR sensor 250, and the motion sensor 270 may be sequentially performed, and determination operations of the processor 220 may be sequentially performed.

According to an embodiment, the operations shown in FIG. 7 may be performed by the processor 220 (e.g., processor 120 of FIG. 1) of the wearable electronic device 200 (e.g., smart watch). For example, the memory 230 (e.g., memory 130 of FIG. 1) of the wearable electronic device 200 (e.g., smart watch) may include instructions that cause the processor 220 to perform at least some operations shown in FIG. 7 when the processor 220 is executed.

According to an embodiment, the operations shown in FIG. 7 may be performed by a processor (e.g., processor 120 of FIG. 1) of an external electronic device (e.g., smart phone, tablet PC, wearable electronic device). For example, memory (e.g., memory 130 of FIG. 1) of the external electronic device (e.g., smart phone, tablet PC, wearable electronic device) may include instructions that cause the processor 120 to perform at least some operations shown in FIG. 7 when the processor 120 is executed.

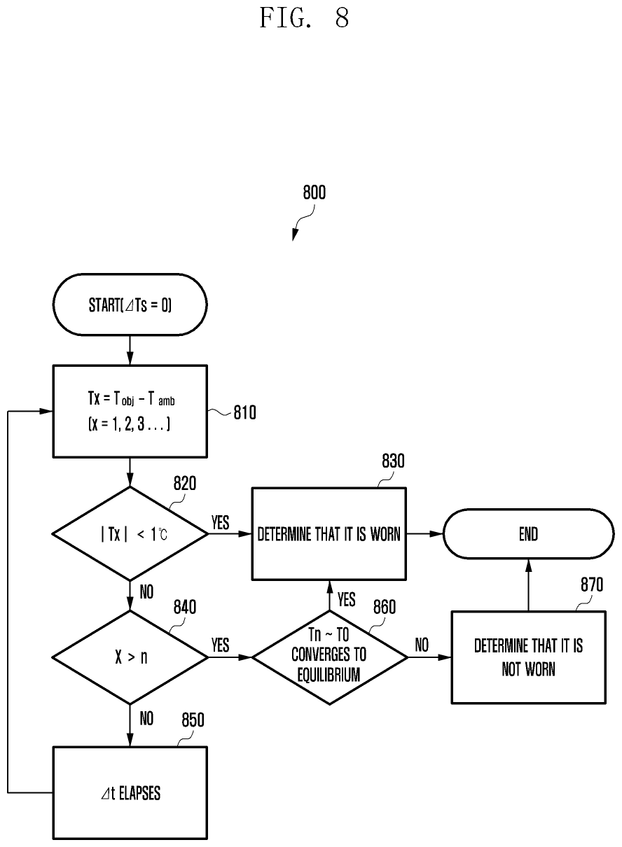

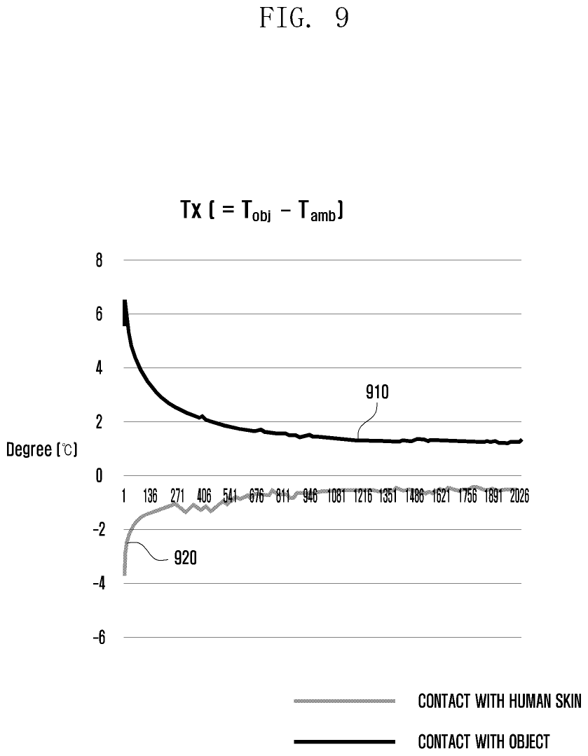

FIG. 8 is a flowchart illustrating a method of determining temperature equilibrium of a wearable electronic device (e.g., smart watch) according to an embodiment of the disclosure. FIG. 9 illustrates a change in the temperature when a wearable electronic device (e.g., smart watch) is in contact with the human skin and in contact with an object according to an embodiment of the disclosure.

Hereinafter, a method of identifying whether the measured temperature (user's skin temperature, body temperature or temperature of object) is in the equilibrium sate in operation 750 of FIG. 7 is described with reference to FIGS. 8 and 9.

Referring to FIGS. 8 and 9, flowchart 800 illustrates that, in operation 810, a processor (e.g., processor 220 of FIG. 2) may operate a temperature sensor (e.g., temperature sensor 260 of FIG. 2). The temperature sensor 260 may operate to measure the temperature of the user's skin or the object. Also, the temperature sensor 260 may operate to measure the temperature of the device (e.g., wearable electronic device 200). The processor 220 may calculate a difference between the measured temperature (e.g., user's skin temperature or temperature of object) and the temperature of the device (e.g., wearable electronic device 200) (Tₓ=T_{obj}−T_{amb}).

In operation 820, the processor 220 may determine whether an absolute value (Tₓ) of difference between the measured temperature (e.g., user's skin temperature or temperature of object) and the temperature of the device (e.g., wearable electronic device 200) is less than the reference temperature (e.g., 1° C.).

For example, as shown in FIG. 9, referring to the difference between a temperature 920 when the wearable electronic device (e.g., wearable electronic device 200 of FIG. 2) is in contact with the human skin and a temperature 910 when it is contact with an object (Tₓ=T_{obj}−T_{amb}), it can be seen that the temperature 920 when the wearable electronic device (e.g., wearable electronic device 200 of FIG. 2) is in contact with the human skin reaches the temperature equilibrium (or converges to temperature equilibrium). If about 30 minutes elapses after the wearable electronic device 200 is worn on the user's body (e.g., wrist), the difference (Tₓ=T_{obj}−T_{amb}) occurs between the temperature 920 when the wearable electronic device 200 is in contact with the human skin and the temperature 910 when it is in contact with the object. If the wearable electronic device 200 is worn on the user's body (e.g., wrist), the skin and the sensor portion come into contact (or close contact) due to the soft nature of the skin although there is a curve in an area in which a sensor portion (e.g., electrode sensor 240, IR sensor 250, temperature sensor 260) is provided. Therefore, when the wearable electronic device 200 is worn on the user's body (e.g., wrist), heat conduction may occur and a state of the temperature equilibrium (or converging to the temperature equilibrium) may be quickly reached.

On the other hand, when the wearable electronic device 200 is in contact with the object, the sensor portion and the object may not be in contact (or in close contact). Therefore, heat conduction (or heat exchange) with the object is slowed, so heat transfer is performed through convection by the surrounding air. Due to this, the state of the temperature equilibrium (or converging to temperature equilibrium) may be reached late (or the temperature does not reach a steady state), and a temperature equilibrium value may also be higher than a reference value.

When an absolute value (|Tₓ|) of a difference between the measured temperature (e.g., user's skin temperature or temperature of object) and the temperature of the device (e.g., wearable electronic device 200) is less than the reference temperature (e.g., about 1° C.) (YES) as a determination result of operation 820, operation 830 may be performed.

In operation 830, the processor 220 may determine that the wearable electronic device 200 is worn on the user's body (e.g., wrist).

Meanwhile, when the absolute value (|Tₓ|) of the difference between the measured temperature (e.g., user's skin temperature or temperature of object) and the temperature of the device (e.g., wearable electronic device 200) is not less than the reference temperature (e.g., about 1° C.) (NO) as a determination result of operation 820, operation 840 may be performed.

In operation 840, the processor 220 may allow a process (X) of measuring and remeasuring the temperature within a preset number of times (n) to be performed. The processor 220 may determine whether the process (X) of measuring and remeasuring the temperature is performed within the preset number of times (n). For example, the processor 220 may measure the temperature n times and connect the values to estimate the trend of temperature change, and may determine whether the values converge to the target temperature. For example, the set number of times may allow remeasurement to be repeatedly performed at regular intervals.

For example, when the process (X) of remeasuring the temperature is performed within the preset number of times (n) (N0), the processor 220 may perform operation 850.

For example, when the process (X) of remeasuring the temperature exceeds the preset number of times (n) (YES), the processor 220 may perform operation 860.

In operation 850, after a predetermined period of time (e.g., 15 minutes, 30 minutes, 60 minutes, 120 minutes) elapses, the processor 220 may allow the operation of measuring the temperature to be reperformed by branching to operation 810.

In operation 860, the processor 220 may determine whether an absolute value (Tₓ) of the difference between the measured (or remeasured) temperature (e.g., user's skin temperature or temperature of object) and the temperature of the device (e.g., wearable electronic device 200) is less than the reference temperature (e.g., 1° C.). When the time has already elapsed, but the temperature difference is still greater than or equal to 1 degree (or when the temperature difference is greater than or equal to 1 degree and has converged), it may represent object measurement.

In operation 850, when the temperature difference is 1 degree or more, but the temperature equilibrium has not reached since a predetermined period of time has not elapsed, the processor 220 may wait for a preset period of time to elapse.

When the absolute value (|Tₓ|) of the difference between the remeasured temperature (e.g., user's skin temperature or temperature of object) and the temperature of the device (e.g., wearable electronic device 200) is not less than the reference temperature (e.g., 1° C.) (NO) as a determination result of operation 860, operation 870 may be performed.

In operation 870, the processor 220 may determine that the wearable electronic device 200 is not worn on the user's body (e.g., wrist).

According to an embodiment, when an absolute value (Tₓ) of a difference between the first measured first temperature (e.g., user's skin temperature or temperature of object) and the temperature of the device (e.g., wearable electronic device 200) is not less than the reference temperature (e.g., 1° C.), the processor 220 may store the absolute value (Tₓ) of the difference between the first temperature (e.g., user's skin temperature or temperature of object) and the temperature of the device (e.g., wearable electronic device 200) as a first temperature value (T1). The processor 220 may allow remeasurement of the temperature to be performed. When an absolute value (Tₓ) of a difference between the second measured second temperature (e.g., user's skin temperature or temperature of object) and the temperature of the device (e.g., wearable electronic device 200) is not less than the reference temperature (e.g., 1° C.), the processor 220 may store the absolute value (Tₓ) of the difference between the second temperature (e.g., user's skin temperature or temperature of object) and the temperature of the device (e.g., wearable electronic device 200) as a second temperature value (T2). The processor 220 may compare the first temperature value (T1) and the second temperature value (T2), and when the second temperature value (T2) is equal to the first temperature value (T1) or when the second temperature value (T2) is greater than the first temperature value (T1), may determine that the temperature equilibrium is not reached (or convergence to the temperature equilibrium is not achieved) and may perform operation 870.

According to an embodiment, as shown in FIG. 9, when the slope is less than or equal to a certain value at points in times Tₓ and Tₓ−1, the processor 220 may determine that the temperature equilibrium is maintained. The processor 220 may record a difference value of temperature in memory (e.g., memory 230 of FIG. 2) (e.g., store data of difference value of temperature), or may record a previous temperature value in the memory 230 (e.g., store data of previous temperature value). The processor 220 may determine the slope of temperature change based on the difference value of temperature and the previous temperature value recorded in the memory 230.

In operation 870, the processor 2200 may determine that the wearable electronic device 200 is not worn on the user's body (e.g., wrist).

Meanwhile, the processor 220 may compare the first temperature value (T1) and the second temperature value (T2) and, when the second temperature value (T2) is less than the first temperature value (T1), may allow an operation of measuring the temperature to be reperformed by branching to operation 810.

For example, in operation 840, the processor 220 may allow the process (X) of measuring and remeasuring the temperature to be performed within the preset number of times (n).

At least some of the operations shown in FIGS. 8 and 9 may be omitted. At least some operations mentioned with reference to other drawings herein may be added and/or inserted before or after at least some operations shown in FIGS. 8 and 9.

According to an embodiment, operations of the electrode sensor 240, the IR sensor 250, and the temperature sensor 260 may be performed in parallel, and determination operations of the processor 220 may be sequentially performed.

According to an embodiment, operations of the electrode sensor 240, the IR sensor 250, and the temperature sensor 260 may be sequentially performed, and determination operations of the processor 220 may be sequentially performed.

According to an embodiment, the operations shown in FIGS. 8 and 9 may be performed by the processor 220 (e.g., processor 120 of FIG. 1) of the wearable electronic device 200 (e.g., smart watch). For example, the memory 230 (e.g., memory 130 of FIG. 1) of the wearable electronic device 200 (e.g., smart watch) may include instructions that cause the processor 220 to perform at least some operations shown in FIGS. 8 and 9 when the processor 220 is executed.

According to an embodiment, the operations shown in FIGS. 8 and 9 may be performed by a processor (e.g., processor 120 of FIG. 1) of an external electronic device (e.g., smart phone, tablet PC, wearable electronic device). For example, memory (e.g., memory 130 of FIG. 1) of the external electronic device (e.g., smart phone, tablet PC, wearable electronic device) may include instructions that cause the processor 120 to perform at least some operations shown in FIGS. 8 and 9 when the processor 120 is executed.

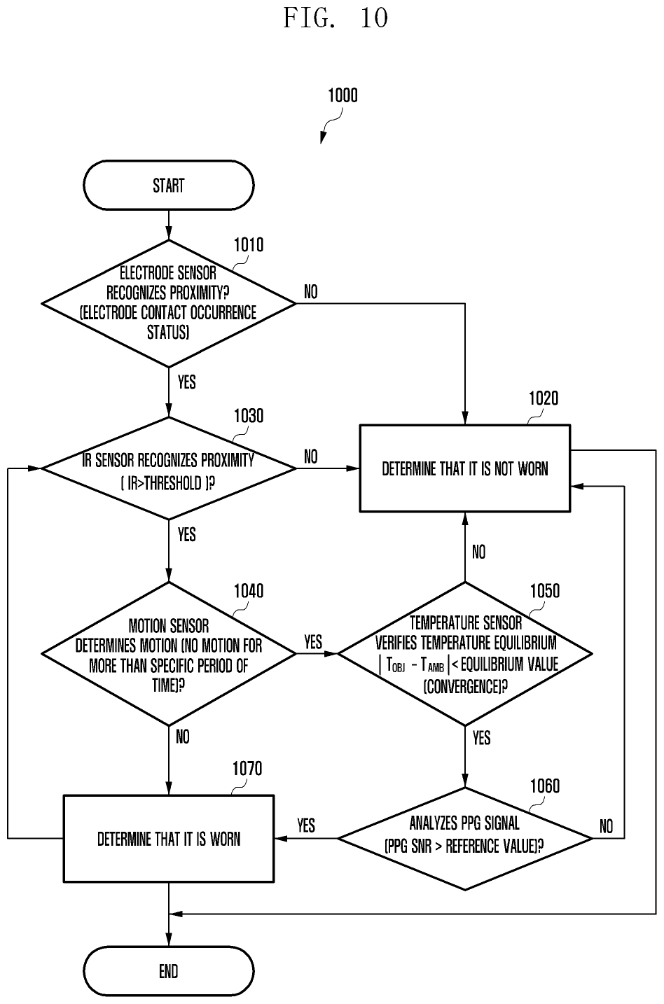

FIG. 10 is a flowchart illustrating a method of operating a wearable electronic device (e.g., smart watch) according to an embodiment of the disclosure.

Referring to FIGS. 2 and 10, flowchart 1000 illustrates that, in operation 1010, a processor 220 may operate an electrode sensor 240. Through operating of the electrode sensor 240, whether the electrode sensor 240 and the user skin are close (or in contact) may be identified. Through operating of the electrode sensor 240, a first biometric signal may be acquired. The processor 220 may determine whether the wearable electronic device 200 is closed to (or in contact with) the skin of the user based on the first biometric signal.

When it is not determined that the wearable electronic device 200 is closed to (or in contact with) the skin of the user (NO) as a determination result of operation 1010, operation 1020 may be performed.

In operation 1020, the processor 220 may determine that the wearable electronic device 200 is not worn on the user's body (e.g., wrist).

When it is determined that the wearable electronic device 200 is close to (or in contact with) the skin of the user (YES) as a determination result of operation 1010, operation 1030 may be performed.

In operation 1030, the processor 220 may operate the IR sensor 250. Through operating of the IR sensor 250, whether the IR sensor 250 and the user skin are close (or in contact) may be identified. Through operating of the IR sensor 250, a second biometric signal may be acquired. The processor 220 may determine whether the wearable electronic device 200 is close to (or in contact with) the skin of the user based on the second biometric signal.

For example, the processor 220 may determine whether the second biometric signal (IR) exceeds the threshold and may determine whether the wearable electronic device 200 is close to (or in contact with) the skin of the user. If the second biometric signal (IR) does not exceed the threshold (NO), the processor 220 may perform operation 1020.

In operation 1020, the processor 220 may determine that the wearable electronic device 200 is not worn on the user's body (e.g., wrist).

If the second biometric signal (IR) exceeds the threshold (YES) as a determination result of operation 1030, the processor 220 may perform operation 1040.

In operation 1040, the processor 220 may operate the motion sensor 270. The motion sensor 270 may sense motion of the wearable electronic device 200, and may acquire a motion sensing signal according to the motion. The processor 220 may determine whether there is no motion for more than a specific period of time (e.g., more than preset period of time) based on the motion sensing signal. For example, when the motion of the wearable electronic device 200 is not detected for more than the specific period of time (e.g., more than preset period of time), the wearable electronic device 200 may not be worn on the user's body (e.g., wrist), but may be placed elsewhere (e.g., placed on table) or may be stored (e.g., stored in drawer or box). In this case, the processor 220 may determine whether the wearable electronic device 200 is worn based on the motion sensing signal.

If the motion of the wearable electronic device 200 is detected within the specific period of time (e.g., within preset period of time) (NO) as a determination result of operation 1040, operation 1070 may be performed.

In operation 1070, the processor 220 may determine that the wearable electronic device 200 is worn on the user's body (e.g., wrist).

If it is determined that there is no motion of the wearable electronic device 200 for more than the specific period of time (e.g., more than preset period of time) (YES) as a determination result of operation 1040, operation 1050 may be performed.

In operation 1050, the processor 220 may operate the temperature sensor 260. Through operating of the temperature sensor 260, a third biometric signal may be acquired.

For example, if the temperature is measured when the user is wearing the wearable electronic device 200, the third biometric signal includes data of the user's skin temperature or body temperature. For example, if the temperature is measured when the user does not wear the wearable electronic device 200, the third biometric signal includes data of the temperature of an object and the ambient temperature of the wearable electronic device 200. The processor 220 may identify whether the equilibrium of the measured temperature (user's skin temperature, body temperature or temperature of object) included in the third biometric signal is achieved. For example, if the equilibrium of the measured temperature (user's skin temperature, body temperature or temperature of object) is not identified based on temperature equilibrium (Tₓ(=T_{obj}−T_{amb})) data when the wearable electronic device 200 comes into contact with the human skin and comes into contact with the object (NO), the processor 220 may perform operation 1020.

In operation 1020, the processor 220 may determine that the wearable electronic device 200 is not worn on the user's body (e.g., wrist).

In operation 1050, the processor 220 may calculate a difference between the measured temperature (user's skin temperature, body temperature or temperature of object) and the device temperature (e.g., temperature of wearable electronic device 200). The processor 220 may determine whether the measured temperature (user's skin temperature, body temperature or temperature of object) is in an equilibrium state (or converges to equilibrium state) based on the difference between the measured temperature (user's skin temperature, body temperature or temperature of object) and the device temperature (e.g., temperature of wearable electronic device 200). If the equilibrium of the measured temperature (user's skin temperature, body temperature or temperature of object) is identified based on temperature equilibrium (Tₓ(=T_{obj}−T_{amb})) data when the wearable electronic device 200 comes into contact with the human skin and comes into contact with the object (YES), the processor 220 may perform operation 1060.

An operation of identifying the equilibrium of the temperature (user's skin temperature, body temperature or temperature of object) performed in operation 1050 may be at least similar or identical to the operation described with reference to FIGS. 8 and 9.

In operation 1060, the processor 220 may operate the IR sensor 250. Through operating of the IR sensor 250, the second biometric signal that includes a photoplethysmogram (PPG) signal may be acquired. The processor 220 may analyze the PPG signal included in the second biometric signal from the IR sensor 250 and may determine whether a PPG SNR value exceeds a reference value.

For example, the processor 220 may identify whether a human-like periodic signal (about 50 to about 200 BPM) is included in the second biometric signal. When the human-like periodic signal (about 50 to about 200 BPM) is included in the second biometric signal, it may be determined that the wearable electronic device 200 is worn on the user's body (e.g., wrist).

For example, when there is no motion of the wearable electronic device 200 for more than the specific period of time (e.g., more than preset period of time), the wearable electronic device 200 may not be worn on the user's body (e.g., wrist), but may be placed elsewhere (e.g., placed on table) or stored (e.g., stored in drawer or box). However, although the user is actually wearing the wearable electronic device 200, there may be no motion for more than the specific period of time (e.g., more than preset period of time) (e.g., sleeping state or no or very small body motion state). In this case, whether the wearable electronic device 200 is being worn on the body (e.g., wrist) may be additionally identified using a PPG SNR value.

If the PPG SNR value does not exceed the reference value as a determination result of operation 1060, operation 1020 may be performed.

In operation 1020, the processor 220 may determine that the wearable electronic device 200 is not worn on the user's body (e.g., wrist). For example, when the PPG SNR value does not exceed the reference value (NO), the wearable electronic device 200 may not be worn on the user's body (e.g., wrist), but may be placed elsewhere or may be stored.

Meanwhile, if the PPG SNR value exceeds the reference value as a determination result of operation 1060, operation 1070 may be performed.

In operation 1070, the processor 220 may determine that the wearable electronic device 200 is worn on the user's body (e.g., wrist).

At least some of the operations shown in FIG. 10 may be omitted. At least some operations mentioned with reference to other drawings herein may be added and/or inserted before or after at least some operations shown in FIG. 10.

According to an embodiment, operations of the electrode sensor 240, the IR sensor 250, the motion sensor 270, and the temperature sensor 260 may be performed in parallel, and determination operations of the processor 220 may be sequentially performed.

According to an embodiment, operations of the electrode sensor 240, the IR sensor 250, the motion sensor 270, and the temperature sensor 260 may be sequentially performed, and determination operations of the processor 220 may be sequentially performed.

According to an embodiment, the operations shown in FIG. 10 may be performed by the processor 220 (e.g., processor 120 of FIG. 1) of the wearable electronic device 200 (e.g., smart watch). For example, the memory 230 (e.g., memory 130 of FIG. 1) of the wearable electronic device 200 (e.g., smart watch) may include instructions that cause the processor 220 to perform at least some operations shown in FIG. 10 when the processor 220 is executed.

According to an embodiment, the operations shown in FIG. 10 may be performed by a processor (e.g., processor 120 of FIG. 1) of an external electronic device (e.g., smart phone, tablet PC, wearable electronic device). For example, memory (e.g., memory 130 of FIG. 1) of the external electronic device (e.g., smart phone, tablet PC, wearable electronic device) may include instructions that cause the processor 120 to perform at least some operations shown in FIG. 10 when the processor 120 is executed.

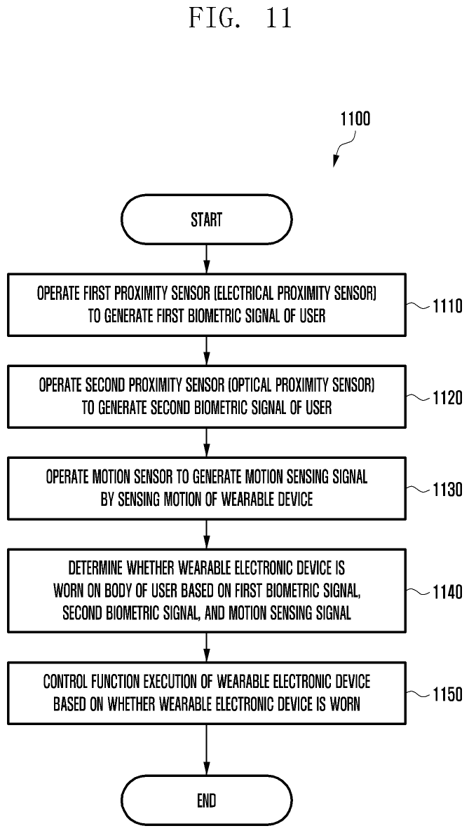

FIG. 11 is a flowchart illustrating a method of operating a wearable electronic device (e.g., smart watch) according to an embodiment of the disclosure.

Referring to FIGS. 2 and 11, flowchart 1100 illustrates that, in operation 1110, a processor 220 may operate a first proximity sensor (e.g., electrode sensor 240, electrical proximity sensor, electrical wearing sensor). The first proximity sensor (e.g., electrode sensor 240, electrical proximity sensor, electrical wearing sensor) may operate to generate a first biometric signal of a user. The processor 220 may acquire the first biometric signal of the user.

In operation 1120, the processor 220 may operate a second proximity sensor (e.g., IR sensor 250, optical proximity sensor, optical wear sensor). The second proximity sensor (e.g., IR sensor 250, optical proximity sensor, optical wear sensor) may operate to generate a second biometric signal of the user. The processor 220 may acquire the second biometric signal.

In operation 1130, the processor 220 may operate the motion sensor 270. The motion sensor 270 may operate to sense motion of the wearable electronic device 200, and to generate a motion sensing signal according to the motion of the wearable electronic device 200. The processor 220 may acquire the motion sensing signal.

In operation 1140, the processor 220 may determine whether the wearable electronic device 200 is worn on a body of the user based on the first biometric signal, the second biometric signal, and the motion sensing signal. Without being limited thereto, the processor 220 may determine whether the wearable electronic device 200 is worn on the body of the user based on at least one of the first biometric signal, the second biometric signal, and the motion sensing signal.

In operation 1150, the processor 220 may control function execution of the wearable electronic device 200 based on whether the wearable electronic device 200 is worn. For example, when the wearable electronic device 200 is worn on the user's body (e.g., wrist), the processor 220 may normally activate (on) functions of the wearable electronic device 200. For example, when the wearable electronic device 200 is not worn on the user's body (e.g., wrist), the processor 220 may deactivate all of or some of functions of the wearable electronic device 200. If all of or some of the functions of the wearable electronic device 200 are deactivated (off), power consumption of the wearable electronic device 200 may be reduced.

At least some of the operations shown in FIG. 11 may be omitted. At least some operations mentioned with reference to other drawings herein may be added and/or inserted before or after at least some operations shown in FIG. 11.

According to an embodiment, operations of the electrode sensor 240, the IR sensor 250, the motion sensor 270, and the temperature sensor 260 may be performed in parallel, and determination operations of the processor 220 may be sequentially performed.

According to an embodiment, operations of the electrode sensor 240, the IR sensor 250, the motion sensor 270, and the temperature sensor 260 may be sequentially performed, and determination operations of the processor 220 may be sequentially performed.

According to an embodiment, the operations shown in FIG. 11 may be performed by the processor 220 (e.g., processor 120 of FIG. 1) of the wearable electronic device 200 (e.g., smart watch). For example, the memory 230 (e.g., memory 130 of FIG. 1) of the wearable electronic device 200 (e.g., smart watch) may include instructions that cause the processor 220 to perform at least some operations shown in FIG. 11 when the processor 220 is executed.

According to an embodiment, the operations shown in FIG. 11 may be performed by a processor (e.g., processor 120 of FIG. 1) of an external electronic device (e.g., smart phone, tablet PC, wearable electronic device). For example, memory (e.g., memory 130 of FIG. 1) of the external electronic device (e.g., smart phone, tablet PC, wearable electronic device) may include instructions that cause the processor 120 to perform at least some operations shown in FIG. 11 when the processor 120 is executed.

An embodiment of the disclosure may normally activate (on) functions of the wearable electronic device 200 when the wearable electronic device 200 is worn on the user's body (e.g., wrist).

An embodiment of the disclosure may deactivate all or some of functions the wearable electronic device 200 when the wearable electronic device 200 is not worn the user's body (e.g., wrist). If all or some of the functions of the wearable electronic device 200 are deactivated (off), power consumption of the wearable electronic device 200 may be reduced.

For example, in the case of the wearable electronic device 200 in which usage time is important, whether the user wears it may be identified and when the user is not wearing the wearable electronic device 200, power consumption may be reduced by deactivating (off) unnecessary functions, such as exercise, health, and notification functions, when the user is not wearing the wearable electronic device 200.

For example, when the user wears the wearable electronic device 200, the processor 220 may detect exercise and may activate (on) a sensor for collecting biometric signals. Through this, it is possible to activate preset various functions, such as the user's heart rate, sleep, and stress

For example, functions of always-on watch and notification delivery while wearing may be activated or deactivated, depending on whether the user is wearing the wearable electronic device 200.

For example, as functions, such as a payment function of the user using the wearable electronic device 200 or collection of personal biometric signals, are added, functions for personal information protection need to be provided. Information regarding whether the wearable electronic device 200 is worn may be a basic element for such functions. Depending on whether the wearable electronic device 200 is worn, it is possible to implement minimum security functions by locking the wearable electronic device 200 to prevent key functions from operating, and to implement more precise safer security functions using this information

A wearable electronic device (e.g., wearable electronic device 200 of FIGS. 2 and 3) according to an embodiment of the disclosure may include an electrical proximity sensor (e.g., electrode sensor 240 of FIG. 2) configured to generate a first biometric signal of a user, an optical proximity sensor (e.g., IR sensor 250 of FIG. 2) configured to generate a second biometric signal of the user, a first temperature sensor configured to generate a third biometric signal by measuring the temperature of the user or an object, a second temperature sensor configured to generate a device temperature signal by measuring the internal temperature of the wearable electronic device, a motion sensor (e.g., motion sensor 270 of FIG. 2) configured to generate a motion sensing signal by sensing motion of the wearable electronic device 200, a processor (e.g., processor 220 of FIG. 2) configured to control operations of the electrode sensor 240, the IR sensor 250, the motion sensor 270, the first temperature sensor, and the second temperature sensor, and memory configured to operatively connect to the processor. When executed, the processor 220 may determine whether the wearable electronic device 200 is worn on a body of the user based on the first biometric signal, the second biometric signal, the third biometric signal, and the motion sensing signal. When executed, the processor 220 may control function execution of the wearable electronic device 200 depending on whether the wearable electronic device 200 is worn.

According to an embodiment, the processor 220 may determine whether the electrode sensor 240 and the skin of the user are close based on the first biometric signal.

According to an embodiment, when the electrode sensor 240 and the skin of the user are not close, the processor 220 may determine that the wearable electronic device 200 is not worn on the body of the user.

According to an embodiment, when the electrode sensor 240 and the skin of the user are close, the processor 220 may determine whether the IR sensor 250 and the skin of the user are close based on the second biometric signal.

According to an embodiment, when the IR sensor 250 and the skin of the user are not close, the processor 220 may determine that the wearable electronic device 200 is not worn on the body of the user.

According to an embodiment, when the electrode sensor 240 and the IR sensor 250 and the skin of the user are close, the processor 220 may identify motion of the wearable electronic device 200 for a preset period of time based on the motion sensing signal. When there is no motion of the wearable electronic device 200 for more than the preset period of time, the processor 220 may determine that the wearable electronic device 200 is not worn on the body of the user.

According to an embodiment, when the motion of the wearable electronic device 200 is detected within the preset period of time, the processor 220 may determine that the wearable electronic device 200 is worn on the body of the user.

According to an embodiment, when there is no motion of the wearable electronic device 200 for more than the preset period of time, the processor 220 may identify whether the measured temperature of the user or the object has reached the temperature equilibrium and may determine whether the user is wearing the wearable electronic device 200.

According to an embodiment, the processor 220 may calculate the equilibrium temperature by subtracting a second temperature value that measures the temperature of the wearable electronic device 200 from a first temperature value that measures the temperature of the user or the object. The processor 220 may compare an absolute value of the equilibrium temperature and the reference value. When a difference between the absolute value of the equilibrium temperature and the reference value is less than a preset value, the processor 220 may determine that the temperature equilibrium is achieved. When the temperature equilibrium is achieved, the processor 220 may determine that the wearable electronic device 200 is worn the body of the user.

According to an embodiment, when a difference between the absolute value of the equilibrium temperature and the reference value is not less than a preset value, the processor 220 may determine that the temperature equilibrium is not achieved. When the temperature equilibrium is not achieved, the processor 220 may determine that the wearable electronic device 200 is not worn on the body of the user.

According to an embodiment, if it is determined that the wearable electronic device 200 is not worn on the body of the user, the processor 220 may deactivate all of or at least some of functions of the wearable electronic device 200.

A method of operating the wearable electronic device 200 according to an embodiment of the disclosure may operate the electrode sensor 240 to generate a first biometric signal of a user. The method may operate the IR sensor 250 to generate a second biometric signal of the user. The method may operate the motion sensor 270 to generate a motion sensing signal by sensing motion of the wearable electronic device 200. The method may operate a first temperature sensor to generate a third biometric signal by measuring the temperature of the user or an object. The method may operate a second temperature sensor to generate a device temperature signal by measuring the internal temperature of the wearable electronic device. The method may determine whether the wearable electronic device 200 is worn on a body of the user based on the first biometric signal, the second biometric signal, the third biometric signal, the device temperature signal, and the motion sensing signal. The method may control function execution of the wearable electronic device 200 depending on whether the wearable electronic device 200 is worn.

According to an embodiment, whether the electrode sensor 240 and the skin of the user are close may be determined based on the first biometric signal. When the skin of the user and the electrode sensor 240 are not close, it may be determined that the wearable electronic device 200 is not worn on the body of the user.

According to an embodiment, when the electrode sensor 240 and the skin of the user are close, whether the skin of the user and the IR sensor 250 are close may be determined based on the second biometric signal.

According to an embodiment, when the IR sensor 250 and the skin of the user are not close, it may be determined that the wearable electronic device 200 is not worn on the body of the user.

According to an embodiment, when the electrode sensor 240 and the IR sensor 250, and the skin of the user are close, motion of the wearable electronic device 200 may be identified for a preset period of time based on the motion sensing signal. When there is no motion of the wearable electronic device 200 for more than the preset period of time, it may be determined that the wearable electronic device 200 is not worn on the body of the user.

According to an embodiment, when the motion of the wearable electronic device 200 is detected within the preset period of time, it may be determined that the wearable electronic device 200 is worn on the body of the user.

According to an embodiment, when there is no motion of the wearable electronic device 200 for more than the preset period of time, whether the measured temperature of the user or the object has reached the temperature equilibrium may be identified. Whether the wearable electronic device 200 is worn may be determined based on whether the measured temperature of the user or the object has reached the temperature equilibrium.

According to an embodiment, the equilibrium temperature may be calculated by subtracting a second temperature value that measures the temperature of the wearable electronic device from a first temperature value that measures the temperature of the user or the object. An absolute value of the equilibrium temperature and the reference value may be compared. When a difference between the absolute value of the equilibrium temperature and the reference value is less than a preset value, it may be determined that the temperature equilibrium is achieved. When the temperature equilibrium is achieved, it may be determined that the wearable electronic device is worn on the body of the user.

According to an embodiment, when the difference between the absolute value of the equilibrium temperature and the reference value is not less than a preset value, it may be determined that the temperature equilibrium is not achieved. When the temperature equilibrium is not achieved, it may be determined that the wearable electronic device 200 is not worn on the body of the user. If it is determined that the wearable electronic device 200 is not worn on the body of the user, all of or at least some of functions of the wearable electronic device 200 may be deactivated.

A recording medium according to an embodiment of the disclosure refers to a recording medium that stores instructions readable by a processor (e.g., processor 120 of FIG. 1, the processor 220 of FIG. 2) of an electronic device, and the instructions, when executed by the processor 120, 220, may allow an operation of operating, by the processor 120, 220, an electrical proximity sensor (e.g., electrode sensor 240 of FIG. 2) to generate a first biometric signal of a user. The instructions, when executed by the processor 120, 220, may allow an operation of operating an optical proximity sensor (e.g., IR sensor 250 of FIG. 2) to generate a second biometric signal of the user to be performed. The instructions, when executed by the processor 120, 220, may allow an operation of operating a motion sensor (e.g., motion sensor 270 of FIG. 2) to generate a motion sensing signal by sensing motion of the wearable electronic device 200 to be performed. The instructions, when executed by the processor 120, 220, may allow an operation of operating a first temperature sensor to generate a third biometric signal by measuring the temperature of the user or an object to be performed. The instructions, when executed by the processor 120, 220, may allow an operation of operating a second temperature sensor to generate a device temperature signal by measuring the internal temperature of the wearable electronic device to be performed. The instructions, when executed by the processor 120, 220, may allow an operation of determining whether the wearable electronic device 200 is worn on the body of the user based on the first biometric signal, the second biometric signal, the third biometric signal, the device temperature signal, and the motion sensing signal may be performed. The instructions, when executed by the processor 120, 220, may allow an operation of controlling function execution of the wearable electronic device 200 depending on whether the wearable electronic device 200 is worn.

The wearable electronic device 200 and a method of operating the same according to an embodiment of the disclosure may reduce misrecognition when determining whether the wearable electronic device 200 is worn and may more precisely determine whether it is worn.

A wearable electronic device (e.g., smart watch) and a method of operating the same according to an embodiment of the disclosure may improve misrecognition of wear detection may reduce an amount of time required to determine whether it is worn.

A wearable electronic device (e.g., smart watch) and a method of operating the same according to an embodiment of the disclosure may improve accuracy of determining whether the wearable electronic device (e.g., smart watch) is worn based on body temperature maintenance and skin temperature change features using a body temperature sensor.

A wearable electronic device (e.g., smart watch) and a method of operating the same according to an embodiment of the disclosure may control function execution by determining whether the electronic device (e.g., smart watch) is worn.

A wearable electronic device (e.g., smart watch) and a method of operating the same according to an embodiment of the disclosure may identify whether the wearable electronic device in which usage time is important is worn and may reduce power consumption by deactivating (off) unnecessary functions, such as exercise, health, and notification functions, when a user is not wearing the wearable electronic device.

In a wearable electronic device (e.g., smart watch) and a method of operating the same according to an embodiment of the disclosure, when a user wears the wearable electronic device, the processor 220 may detect exercise and may activate (on) a sensor for collecting biometric signals. Through this, it is possible to activate preset various functions, such as the user's heart rate, sleep, and stress.

A wearable electronic device (e.g., smart watch) and a method of operating the same according to an embodiment of the disclosure may activate or deactivate functions of always-on watch and notification delivery while wearing, depending on whether the wearable electronic device is worn.

A wearable electronic device (e.g., smart watch) and a method of operating the same according to an embodiment of the disclosure may implement functions for personal information protection using information regarding whether the wearable electronic device is worn. Depending on whether the wearable electronic device is worn, it is possible to implement minimum security functions by locking the wearable electronic device to prevent key functions from operating, and to implement more precise and safer security functions using this information.

The electronic device according to various embodiments may be one of various types of electronic devices. The electronic devices may include, for example, a portable communication device (e.g., a smartphone), a computer device, a portable multimedia device, a portable medical device, a camera, a wearable electronic device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a compiler or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device individually or collectively, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A wearable electronic device comprising:
an electrical proximity sensor configured to generate a first biometric signal of a user;
an optical proximity sensor configured to generate a second biometric signal of the user;
a motion sensor configured to generate a motion sensing signal by sensing motion of the wearable electronic device;
a first temperature sensor configured to generate a third biometric signal by measuring a temperature of the user or an object;
a second temperature sensor configured to generate a device temperature signal by measuring an internal temperature of the wearable electronic device;
memory storing one or more computer programs; and
one or more processors communicatively coupled to the electrical proximity sensor, the optical proximity sensor, the motion sensor, the first temperature sensor, and the second temperature sensor,
wherein the one or more computer programs include computer-executable instructions that, when executed by the one or more processors individually or collectively, cause the wearable electronic device to:
determine whether the wearable electronic device is worn on a body of the user based on the first biometric signal, the second biometric signal, the third biometric signal, and the motion sensing signal, and
control a function execution of the wearable electronic device based on whether the wearable electronic device is worn.

2. The wearable electronic device of claim 1, wherein the one or more computer programs further include computer-executable instructions that, when executed by the one or more processors individually or collectively, cause the wearable electronic device to:
determine whether the electrical proximity sensor and skin of the user are close based on the first biometric signal.

3. The wearable electronic device of claim 2, wherein, when the electrical proximity sensor and the skin of the user are determined to not be close, the one or more computer programs further include computer-executable instructions that, when executed by the one or more processors individually or collectively, cause the wearable electronic device to:
determine that the wearable electronic device is not worn on the body of the user.

4. The wearable electronic device of claim 2, wherein, when the electrical proximity sensor and the skin of the user are determined to be close, the one or more computer programs further include computer-executable instructions that, when executed by the one or more processors individually or collectively, cause the wearable electronic device to:
determine whether the optical proximity sensor and the skin of the user are close based on the second biometric signal.

5. The wearable electronic device of claim 4, wherein, when the optical proximity sensor and the skin of the user are determined to not be close, the one or more computer programs further include computer-executable instructions that, when executed by the one or more processors individually or collectively, cause the wearable electronic device to:
determine that the wearable electronic device is not worn on the body of the user.

6. The wearable electronic device of claim 4, wherein, when the optical proximity sensor and the skin of the user are determined to be close, the one or more computer programs further include computer-executable instructions that, when executed by the one or more processors individually or collectively, cause the wearable electronic device to:
verify motion of the wearable electronic device for a preset period of time based on the motion sensing signal, and
when there is no motion of the wearable electronic device for more than the preset period of time, determine that the wearable electronic device is not worn on the body of the user.

7. The wearable electronic device of claim 6, wherein, when the motion of the wearable electronic device is detected within the preset period of time, the one or more computer programs further include computer-executable instructions that, when executed by the one or more processors individually or collectively, cause the wearable electronic device to:
determine that the wearable electronic device is worn on the body of the user.

8. The wearable electronic device of claim 6, wherein, when there is no motion of the wearable electronic device for more than the preset period of time, the one or more computer programs further include computer-executable instructions that, when executed by the one or more processors individually or collectively, cause the wearable electronic device to:
verify whether the temperature of the user or the object has reached temperature equilibrium, and
determine whether the user is wearing the wearable electronic device.

9. The wearable electronic device of claim 8, wherein the one or more computer programs further include computer-executable instructions that, when executed by the one or more processors individually or collectively, cause the wearable electronic device to:
calculate an equilibrium temperature by subtracting a second temperature value that measures the temperature of the wearable electronic device from a first temperature value that measures the temperature of the user or the object,
compare an absolute value of the equilibrium temperature and a reference value,
when a difference between the absolute value of the equilibrium temperature and the reference value is less than a preset value, determine that the temperature equilibrium is achieved, and
when it is determined that the temperature equilibrium is achieved, determine that the wearable electronic device is worn on the body of the user.

10. The wearable electronic device of claim 9, wherein the one or more computer programs further include computer-executable instructions that, when executed by the one or more processors individually or collectively, cause the wearable electronic device to:
when a difference between the absolute value of the equilibrium temperature and the reference value is not less than the preset value, determine that the temperature equilibrium is not achieved, and
when it is determined that the temperature equilibrium is not achieved, determine that the wearable electronic device is not worn on the body of the user.

11. The wearable electronic device of claim 1, wherein the one or more computer programs further include computer-executable instructions that, when executed by the one or more processors individually or collectively, cause the wearable electronic device to:
if it is determined that the wearable electronic device is not worn on the body of the user, deactivate all functions or at least some functions of the wearable electronic device.

12. A method performed by a wearable electronic device, the method comprising:
operating, by the wearable electronic device, an electrical proximity sensor to generate a first biometric signal of a user;
operating, by the wearable electronic device, an optical proximity sensor to generate a second biometric signal of the user;
operating, by the wearable electronic device, a motion sensor to generate a motion sensing signal by sensing motion of the wearable electronic device;
operating, by the wearable electronic device, a first temperature sensor to generate a third biometric signal by measuring a temperature of the user or an object;
operating, by the wearable electronic device, a second temperature sensor to generate a device temperature signal by measuring an internal temperature of the wearable electronic device;
determining, by the wearable electronic device, whether the wearable electronic device is worn on a body of the user based on the first biometric signal, the second biometric signal, the third biometric signal, the device temperature signal, and the motion sensing signal; and
controlling, by the wearable electronic device, a function execution of the wearable electronic device based on whether the wearable electronic device is worn.

13. The method of claim 12, further comprising:
determining, by the wearable electronic device, whether the electrical proximity sensor and skin of the user are close based on the first biometric signal; and
based on determining that the electrical proximity sensor and the skin of the user are not close, determining, by the wearable electronic device, that the wearable electronic device is not worn on the body of the user.

14. The method of claim 13, further comprising:
based on determining that the electrical proximity sensor and the skin of the user are close, determining, by the wearable electronic device, whether the optical proximity sensor and the skin of the user are close based on the second biometric signal.

15. The method of claim 14, further comprising:
based on determining that the optical proximity sensor and the skin of the user are not close, determining, by the wearable electronic device, that the wearable electronic device is not worn on the body of the user.

16. The method of claim 14, further comprising:
based on determining that the optical proximity sensor and the skin of the user are close, verifying, by the wearable electronic device, motion of the wearable electronic device for a preset period of time based on the motion sensing signal; and
based on detecting no motion of the wearable electronic device for more than the preset period of time, determining, by the wearable electronic device, that the wearable electronic device is not worn on the body of the user.

17. The method of claim 16, further comprising:
based on detecting the motion of the wearable electronic device within the preset period of time, determining, by the wearable electronic device, that the wearable electronic device is worn on the body of the user.

18. The method of claim 16, further comprising:
based on detecting no motion of the wearable electronic device for more than the preset period of time, verifying, by the wearable electronic device, whether the temperature of the user or the object has reached temperature equilibrium; and
determining, by the wearable electronic device, whether the user is wearing the wearable electronic device.

19. One or more non-transitory computer-readable storage media storing one or more computer programs including computer-executable instructions that, when executed by one or more processors of a wearable electronic device individually or collectively, cause the wearable electronic device to perform operations, the operations comprising:
operating, by the wearable electronic device, an electrical proximity sensor to generate a first biometric signal of a user;
operating, by the wearable electronic device, an optical proximity sensor to generate a second biometric signal of the user;
operating, by the wearable electronic device, a motion sensor to generate a motion sensing signal by sensing motion of the wearable electronic device;
operating, by the wearable electronic device, a first temperature sensor to generate a third biometric signal by measuring a temperature of the user or an object;
operating, by the wearable electronic device, a second temperature sensor to generate a device temperature signal by measuring an internal temperature of the wearable electronic device;
determining, by the wearable electronic device, whether the wearable electronic device is worn on a body of the user based on the first biometric signal, the second biometric signal, the third biometric signal, the device temperature signal, and the motion sensing signal; and
controlling, by the wearable electronic device, a function execution of the wearable electronic device based on whether the wearable electronic device is worn.

20. The one or more non-transitory computer-readable storage media of claim 19, the operations further comprising:
determining, by the wearable electronic device, whether the electrical proximity sensor and skin of the user are close based on the first biometric signal; and
based on determining that the electrical proximity sensor and the skin of the user are not close, determining, by the wearable electronic device, that the wearable electronic device is not worn on the body of the user.
